(19) United States
(12) Patent Application Publication
(10) Pub. No.: US 2024/0084400 A1
(43) Pub. Date: Mar. 14, 2024

(54) METHODS FOR PROGNOSING, DIAGNOSING, AND TREATING COLORECTAL CANCER

(71) Applicant: BOARD OF REGENTS, THE UNIVERSITY OF TEXAS SYSTEM, Austin, TX (US)

(72) Inventors: Eduardo VILAR SANCHEZ, Houston, TX (US); Wenhui WU, Houston, TX (US); Hiroyuki KATAYAMA, Houston, TX (US); Samir HANASH, Houston, TX (US); Prashant V. BOMMI, Houston, TX (US)

(73) Assignee: BOARD OF REGENTS, THE UNIVERSITY OF TEXAS SYSTEM, Austin, TX (US)

(21) Appl. No.: 18/547,495

(22) PCT Filed: Feb. 22, 2022
(86) PCT No.: PCT/US2022/017324

§ 371 Date: Aug. 22, 2023

Related U.S. Application Data
(60) Provisional application No. 63/152,751, filed on Feb. 23, 2021.

(51) Int. Cl. C12Q 1/6886; G01N 33/574

(52) U.S. Cl. CPC C12Q 1/6886; G01N 33/57419; C12Q 2600/158; G01N 2800/50; G01N 2800/52

(57) ABSTRACT

The current disclosure describes effective therapeutic treatments and diagnostic/prognostic methods for colorectal cancer based on the expression or activity level of biomarkers. Aspects of the disclosure relate to a method for treating a subject for colorectal cancer, the method comprising treating the subject for colorectal cancer after the expression level of one or more biomarker genes from Table 1, Table 2, Table 3, Table 4, Table 5, Table S2, Table S3, Table S4, Table S5, FIG. 4A, and/or FIG. 4B has been determined in a sample from the subject.

This application claims benefit of priority of U.S. Provisional Application No. 63/152,751, filed Feb. 23, 2021, which is hereby incorporated by reference in its entirety.

This invention was made with government support under grant number CA219463 awarded by the National Institutes of Health. The government has certain rights in the invention.

### I. FIELD OF THE INVENTION

The present invention relates generally to the fields of molecular biology and oncology. More particularly, it concerns methods and compositions involving cancer prognosis, diagnosis and treatment.

### II. BACKGROUND

Colorectal cancer (CRC) is one of the most frequently diagnosed malignancies and a leading cause of cancer-related deaths worldwide. High degree of mortality associated with CRC is largely due to late disease detection and lack of availability of adequate prognostic biomarkers, including the currently used tumor-node-metastasis (TNM) classification system from the American Joint Committee on Cancer for predicting tumor prognosis and recurrence. This highlights the need to develop robust prognostic biomarkers for CRC, and the expectations are that such biomarkers must offer a superior prognostic clinical usefulness compared to existing TNM staging classification.

### SUMMARY OF THE INVENTION

The current disclosure fulfills a need in the art by providing more effective therapeutic treatments and diagnostic/prognostic methods for colorectal cancer based on the expression or activity level of biomarkers. Aspects of the disclosure relate to a method for treating a subject for colorectal cancer, the method comprising treating the subject for colorectal cancer after the expression level of one or more biomarker genes from Table 1, Table 2, Table 3, Table 4, Table 5, Table S2, Table S3, Table S4, Table S5, FIG. 4A, and/or FIG. 4B has been determined in a sample from the subject. Further aspects relate to a method for evaluating a subject comprising measuring the level of expression of one or more biomarkers of Table 1, Table 2, Table 3, Table 4, Table 5, Table S2, Table S3, Table S4, Table S5, FIG. 4A, and/or FIG. 4B in a sample from the subject. Yet further aspects relate to a method of prognosing and/or diagnosing a subject with colorectal cancer comprising: a) measuring the level of expression of one or more biomarker genes from Table 1, Table 2, Table 3, Table 4, Table 5, Table S2, Table S3, Table S4, Table S5, FIG. 4A, and/or FIG. 4B in a sample from the subject; b) comparing the level(s) of expression to a control sample(s) or control level(s) of expression; and, c) prognosing and/or diagnosing the subject based on the levels of measured expression. Also described are kits comprising 1, 2, 3, 4, or 5 detection agents for determining expression levels of biomarkers for colorectal cancer, wherein the biomarkers comprise one or more biomarker genes from Table 1, Table 2, Table 3, Table 4, Table 5, Table S2, Table S3, Table S4, Table S5, FIG. 4A, and/or FIG. 4B.

In some aspects, at least 5 biomarkers from Table 1, Table 2, Table 3, Table 4, Table 5, Table S2, Table S3, Table S4, and/or Table S5 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 1, Table 2, Table 3, Table 4, Table 5, Table S2, Table S3, Table S4, and/or Table S5 has been determined, is evaluated, or is measured in the methods of the disclosure.

In some aspects, at least 5 biomarkers from Table 1 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 1 has been determined, is evaluated, or is measured in the methods of the disclosure. In some aspects, at least 5 biomarkers from Table 2 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 2 has been determined, is evaluated, or is measured in the methods of the disclosure. In some aspects, at least 5 biomarkers from Table 3 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 3 has been determined, is evaluated, or is measured in the methods of the disclosure. In some aspects, at least 5 biomarkers from Table 4 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 4 has been determined, is evaluated, or is measured in the methods of the disclosure. In some aspects, at least 5 biomarkers from Table 5 has been determined, is evaluated, or is measured in the methods of the disclosure. In some aspects, at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) biomarkers from Table 5 has been determined, is evaluated, or is measured in the methods of the disclosure.

In some aspects, at least Sppl was determined, is evaluated, or is measured in a sample from the subject. In some aspects, at least Mup22 was determined, is evaluated, or is measured in a sample from the subject. In some aspects, at least Slc26a9 was determined, is evaluated, or is measured in a sample from the subject. In some aspects, at least Muc6 was determined, is evaluated, or is measured in a sample from the subject. In some aspects, at least Ugt8a was determined, is evaluated, or is measured in a sample from the subject. In some aspects, at least Meg3 was determined, is evaluated, or is measured in a sample from the subject. In some aspects, at least P2ry4 was determined, is evaluated, or is measured in a sample from the subject. In some aspects, at least Defa5 was determined, is evaluated, or is measured in a sample from the subject. In some aspects, at least Gm49320 was determined, is evaluated, or is measured in a sample from the subject.

The methods of the disclosure may comprise or further comprise calculating a prognosis score. In some aspects, the methods comprise or further comprise determining or calculating a H-score. An H-score can be calculated by determining the intensity of staining of a biomarker protein in an IHC sample. For example, the intensity can be scored as 0, negative; 1+, weak; 2+, moderate; 3+, strong. The percentage of positively stained cells (0 to 100) can be multiplied by the staining intensity score (0/1/2/3), thus yielding scores from 0 to 300. This is further described in Eremo et al., Sci Rep. 2020; 10(1):1451, which is herein incorporated by reference. In some aspects, the subject is treated after determined to have a H-score of greater than 100. In some aspects, the subject is treated after determined to have a H score of at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300, or any derivable range therein. In some aspects, the subject is diagnosed or prognosed with cancer, as having cancer, and/or as having adenocarcinoma after the subject has been determined to have a H-score of greater than 100. In some aspects, the subject is diagnosedor prognosed with cancer, as having cancer, and/or as having adenocarcinoma after the subject has been determined to have a H score of at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300, or any derivable range therein. The methods may include determining, evaluating, or measuring the biomarker protein by immunological detection such as immunofluorescence and/or immunohistochemistry.

In some aspects, the subject has been determined to have Lynch Syndrome. In some aspects, the subject has Lynch Syndrome. In some aspects, the subject has not been diagnosed with Lynch Syndrome. In some aspects, the subject has not been diagnosed with and/or has not been treated for colorectal cancer. In some aspects, the subject has been diagnosed with colorectal cancer. In some aspects, the the subject is treated for stage I or stage II colorectal cancer. In some aspects, the colorectal cancer comprises mismatch repair deficient colorectal cancer (MMR-d). The cancer may be further defined as recurrent cancer, metastatic cancer, or refractory cancer.

The expression level of the biomarker may be a normalized expression level. The sample from the subject may be a sample from a primary colorectal cancer tumor. In some aspects, the sample from the subject is a sample from a biopsy of colorectal tissues. In some aspects, the sample from the subject is a sample of colon mucosa or a culture of cells derived from the colon mucosa of the subject. In some aspects, the sample comprises an organoid derived from the colon mucosa of the subject. In some aspects, the sample comprises tissue samples. In some aspects, the sample comprises formalin fixed paraffin embedded samples (FFPE). In some aspects, the expression levels of the one or more biomarkers in the subject is, was determined to be, is evaluated as, or is measured to be i) increased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk. In some aspects, the expression levels of the one or more biomarkers in the subject is, was determined to be, is evaluated as, or is measured to be i) decreased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk. In some aspects, the subject is treated for colorectal cancer when the expression level of the biomarker gene is increased or decreased according to Tables 3-5. In some aspects, the subject is treated or is prognosed or diagnosed as having cancer, Lynch Syndrome, colorectal cancer and/or a MMR-d cancer when the expression levels of the one or more biomarker genes is decreased or increased, according to Tables 3-5, compared to a control level of expression, wherein the control level of expression is representative of the level of expression of the biomarker gene in samples from subjects identified as not having MMR-d colorectal cancer, identified as low risk, or in normal tissues. In some aspects, the subject is treated or is prognosed or diagnosed as having cancer, Lynch Syndrome, colorectal cancer and/or a MMR-d cancer when the expression levels of the one or more biomarker genes is within the range of expression levels in samples of subjects identified as having MMR-d colorectal cancer or identified as high risk. In some aspects, the subject is not treated or is prognosed or diagnosed as not having cancer, Lynch Syndrome, colorectal cancer and/or a MMR-d cancer when the expression levels of the one or more biomarker genes is not significantly different than the level of expression of the biomarker gene that is representative of the level of expression in samples from subjects identified as not having MMR-d colorectal cancer, identified as low risk, or in normal tissues.

In some aspects, the treatment comprises one or more of surgery, partial colectomy, surgical removal of lymph nodes, radiation, targeting therapy, adjuvant chemotherapy, and neo adjuvant chemotherapy. In some aspects, the treatment excludes one or more of surgery, partial colectomy, surgical removal of lymph nodes, radiation, targeting therapy, adjuvant chemotherapy, and neo-adjuvant chemotherapy. In some aspects, the treatment excludes chemotherapy, adjuvant chemotherapy, or neo-adjuvant chemotherapy. In some aspects, the chemotherapy comprises one or more of 5-FU, leucovorin, oxaliplatin, capecitabine, and irinotecan. In some aspects, the targeted therapy comprises one or more of bevacizumab, ziv-aflibercept, ramucirumab, cetuximab, and panitumumab. In some aspects, the targeted therapy excludes one or more of bevacizumab, ziv-aflibercept, ramucirumab, cetuximab, and panitumumab. In some aspects, the treatment comprises one or more of regorafenib, trifluridine, and tipiracil. In some aspects, the treatment excludes one or more of regorafenib, trifluridine, and tipiracil.

In some aspects, the expression level of the biomarker has been determined, is evaluated, or is measured in the subject by determining, evaluating, or measuring the amount of protein produced from the biomarker gene. In some aspects, the expression level of the biomarker has been determined, is evaluated, or is measured in the subject by determining the amount of mRNA produced from the biomarker gene. In some aspects, the subject has undergone surgery to resect all or part of the cancer. In some aspects, the subject has not undergone surgical resection of the tumor. In some aspects, the level of expression of one of more of the biomarker genes was determined pre-operative and/or post-operative. In some aspects, low risk is indicative of a subject with a low risk for distant metastasis and good overall survival (OS) rate, and high risk is indicative of a subject with a high risk for distant metastasis and poor overall survival (OS) rate.

In some aspects, the method further comprises evaluating the sample from the subject for MMR-d and/or microsatellite instability (MSI). In some aspects, the MMR-d and/or MSI has been evaluated in the subject. In some aspects, evaluating the sample from the subject for MMR-d comprises determining the level of expression of one or more of MLH1, MSH2, MSH6, PMS2, and EPCAM. In some aspects, the subject has been determined to be MMR-d and/or MSI+. In some aspects, the cancer comprises stage 0, I, II, III, or IV cancer. In some aspects, the cancer excludes stage 0, I, II, III, or IV cancer.

In some aspects, the methods exclude determining, measuring, or evaluating one or more biomarker genes from Table 1, Table 2, Table 3, Table 4, Table 5, Table S2, Table S3, Table S4, Table S5, FIG. 4A, and/or FIG. 4B.

Kit aspects of the disclosure may comprise one or more negative or positive control samples and/or control detection agents.

Some aspects further involve isolating nucleic acids such as ribonucleic or RNA from a biological sample or in a sample of the patient. Other steps may or may not include amplifying a nucleic acid in a sample and/or hybridizing one or more probes to an amplified or non-amplified nucleic acid. The methods may further comprise assaying nucleic acids in a sample. Further aspects include isolating or analyzing protein expression in a biological sample for the expression of the biomarker. In certain aspects, a microarray may be used to measure or assay the level of the biomarkers in a sample. The methods may further comprise recording the biomarker expression or activity level in a tangible medium or reporting the expression or activity level to the patient, a health care payer, a physician, an insurance agent, or an electronic system.

The expression level or activity level from a control sample may be an average value, a normalized value, a cut-off value, or an average normalized value. The expression level or activity level may be an average or mean obtained from a significant proportion of patient samples. The expression or activity level may also be an average or mean from one or more samples from the patient.

In some aspects, the elevated level/increased expression or reduced level/decreased expression is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the control, or any derivable range therein. In some aspects, a level of expression may be qualified as “low” or “high,” which indicates the patient expresses a certain gene at a level relative to a reference level or a level with a range of reference levels that are determined from multiple samples meeting particular criteria. The level or range of levels in multiple control samples is an example of this. In some aspects, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, the threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900,1000, 1100, 1200, 1300, 1400, 1500, 1600, 1700,1800, 1900, 2000 or more (or any range derivable therein).

In some aspects, the control may be the average level of expression of the biomarker in a biological sample from a subject having colorectal cancer or determined to be at risk for colorectal cancer. The control may be the level of expression of the biomarker gene in a biological sample from a subject with stage 0, I, II, III, or IV cancer (or any TMN stage defined herein). One skilled in the art would understand that, when comparing the expression level of the miRNA in a biological sample from a test subject to the expression level from a subject with colorectal cancer, the decision to treat the subject for colorectal cancer or diagnose or provide a prognosis that the subject has or is likely to get cancer is based on the a level of expression that is similar to the control or within 1, 2, 3, 4, or 5 deviations or differs by less than 1, 3, 5, 10, 15, 20, 30, or 40% (or any derivable range therein).

In some aspects, the prognosis score is expressed as a number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 5 86, 87, 88, 89, 90, 91, 92, 93, 94, 95, 96, 97, 98, 99, or 100% likelihood (or any range derivable therein) that a patient has a chance of poor survival or cancer recurrence or poor response to a particular treatment. Alternatively, the probability may be expressed generally in percentiles, quartiles, or deciles.

A difference between or among weighted coefficients or expression or activity levels or between or among the weighted comparisons may be, be at least or be at most about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 times or -fold (or any range derivable therein).

In some aspects, determination of calculation of a diagnostic, prognostic, or risk score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain aspects, the prognosis score is calculated using one or more statistically significantly differentially expressed biomarkers (either individually or as difference pairs), including expression or activity levels in a biomarker, gene, or protein.

In some aspects, the biological sample from the patient is a sample from a primary colon cancer tumor. In some aspects, the biological sample is from a tissue or organ as described herein. In still further aspects, the method may comprise obtaining a sample of the subject or patient. Non-limiting examples of the sample include a tissue sample, a whole blood sample, a urine sample, a fecal sample, a biopsy sample, a polyp, a saliva sample, a serum sample, a plasma sample, or a fecal sample.

The methods of obtaining a sample of the subject or patient provided herein include methods of biopsy such as fine needle aspiration, core needle biopsy, vacuum assisted biopsy, incisional biopsy, excisional biopsy, punch biopsy, shave biopsy or skin biopsy.

In certain aspects the sample is obtained from a biopsy from the colon, a polyp, or other associated colonic tissues. In other aspects the sample may be obtained from any of the tissues provided herein that include but are not limited to gall bladder, skin, heart, lung, breast, pancreas, liver, muscle, kidney, smooth muscle, bladder, intestine, brain, prostate, esophagus, or thyroid tissue.

In certain aspects the sample is obtained from cystic fluid or fluid derived from a polyp, tumor, or neoplasm. In yet other aspects the cyst, tumor or neoplasm is in the digestive system. In certain aspects of the current methods, any medical professional such as a doctor, nurse or medical technician may obtain a biological sample for testing. In further aspects of the current methods, the patient or subject may obtain a biological sample for testing without the assistance of a medical professional, such as obtaining a whole blood sample, a urine sample, a fecal sample, a buccal sample, or a saliva sample.

In further aspects, the sample may be a fresh, frozen or preserved sample or a fine needle aspirate. In particular aspects, the sample is a formalin-fixed, paraffin embedded (FFPE) sample. An acquired sample may be placed in short term or long term storage by placing in a suitable medium, excipient, solution, or container. In certain cases storage may require keeping the sample in a refrigerated, or frozen environment. The sample may be quickly frozen prior to storage in a frozen environment. In certain instances the frozen sample may be contacted with a suitable cryopreservation medium or compound. Examples of cryopreservation mediums or compounds include but are not limited to: glycerol, ethylene glycol, sucrose, or glucose.

TABLE 3

| Biomarker | Expression | Biomarker | Expression | Biomarker | Expression |
|---|---|---|---|---|---|
| Spp1 | Increased | Ak4 | Increased | Rpl10a | Increased |
| Nr1h5 | Increased | Sema6a | Increased | Rpl17 | Increased |
| Rian | Increased | Slc28a1 | Increased | Rpl30 | Increased |
| Jchain | Increased | Itga7 | Increased | Rplp1 | Increased |
| Meg3 | Increased | Acsl5 | Increased | S100a11 | Increased |
| Gm28230 | Increased | Actb | Increased | Smarce1 | Increased |
| Dgkh | Increased | Actc1 | Increased | Sult1a1 | Increased |
| Arhgap45 | Increased | Adh1 | Increased | Sumo2 | Increased |
| Trpv3 | Increased | Ahcy | Increased | Tsta3 | Increased |
| P2ry4 | Increased | Ahsa1 | Increased | Ugdh | Increased |
| Ccn3 | Increased | Akr1c12 | Increased | Ugp2 | Increased |
| B230206H07Rik | Increased | Anxa2 | Increased | Ugt1a6b | Increased |
| Tchh | Increased | Apeh | Increased | Ugt2b34 | Increased |
| Lifr | Increased | Arl1 | Increased | Vdac2 | Increased |
| Ahnak | Increased | Atp2a2 | Increased | Vdac3 | Increased |
| Sardh | Increased | Bzw1 | Increased | Wdr1 | Increased |
| Cacna1h | Increased | Ca1 | Increased | Acad9 | Increased |
| Sned1 | Increased | Casp3 | Increased | Adal | Increased |
| Nlrp9b | Increased | Cct6a | Increased | Akr1c13 | Increased |
| Gm23547 | Decreased | Ces1f | Increased | Aldoart1 | Increased |
| Muc5ac | Increased | Clic1 | Increased | Anp32a | Increased |
| Defa5 | Decreased | Ddx39b | Increased | Atad3 | Increased |
| Gm49320 | Decreased | Ddx5 | Increased | Bag1 | Increased |
| Mup22 | Increased | Eif5a | Increased | Banf1 | Increased |
| Slc26a9 | Increased | Fabp6 | Increased | Camk1d | Increased |
| Muc6 | Increased | Fam98b | Increased | Cbx1 | Increased |
| Ugt8a | Increased | Gapdh | Increased | Copg1 | Increased |
| Cubn | Increased | Gm5039 | Increased | Csnk1a1 | Increased |
| Pgc | Increased | Gm7293 | Increased | Ddx17 | Increased |
| Aqp5 | Increased | Gnb2 | Increased | Dynlrb1 | Increased |
| Cyp2c55 | Increased | Gsn | Increased | Eif3c | Increased |
| Abca12 | Increased | Hist1h1t | Increased | Fasn | Increased |
| Slc5a4a | Increased | Hist1h4a | Increased | Gmps | Increased |
| Mal | Increased | Hmgcll1 | Increased | Grb2 | Increased |
| Car1 | Increased | Hmgcs2 | Increased | H2afy | Increased |
| G6pc | Increased | Hsd17b10 | Increased | Hnrnpa0 | Increased |
| Sprr1a | Increased | Hsd17b11 | Increased | Hnrnph2 | Increased |
| Slc5a12 | Increased | Kras | Increased | Ilf3 | Increased |
| Scara3 | Increased | Krt71 | Increased | Lsm14a | Increased |
| Gif | Increased | Krt84 | Increased | Mbnl1 | Increased |
| Slc30a10 | Increased | Lamtor3 | Increased | Mycbp | Increased |
| Lct | Increased | Ldha | Increased | Nudt21 | Increased |
| Sptssb | Increased | Lgals3 | Increased | Nudt5 | Increased |
| Sgk2 | Increased | Mat2a | Increased | Pabpc4 | Increased |
| Clca4a | Increased | Mettl7a1 | Increased | Pafah1b3 | Increased |
| Cyp3a25 | Increased | Mfsd2b | Increased | Pak2 | Increased |
| Gdpd2 | Increased | Mp68 | Increased | Parp1 | Increased |
| Gkn1 | Increased | Mpst | Increased | Pfdn2 | Increased |
| Tmigd1 | Increased | Mptx1 | Increased | Pgm2 | Increased |
| Slc2a2 | Increased | Mydgf | Increased | Prpf19 | Increased |
| Aldh1a1 | Increased | Ncoa4 | Increased | Psmc4 | Increased |
| Fa2h | Increased | Ndufa4 | Increased | Psmd8 | Increased |
| Bst1 | Increased | Ndufb4 | Increased | Rnps1 | Increased |
| 1810065E05Rik | Increased | Ndufb5 | Increased | Rpa3 | Increased |
| Anxa10 | Increased | Nudt9 | Increased | Rpl13a | Increased |
| Slc10a2 | Increased | Pabpc5 | Increased | Rpl13a-ps1 | Increased |
| Clu | Increased | Pitrm1 | Increased | Rpl23a | Increased |
| 2010109I03Rik | Increased | Ppa2 | Increased | Rpl38 | Increased |
| Pdzk1 | Increased | Psma3 | Increased | Rps18 | Increased |
| Cyp2b10 | Increased | Psmd11 | Increased | Rsl1d1 | Increased |
| Mafb | Increased | Qdpr | Increased | Sec13 | Increased |
| Slc5a4b | Increased | Rab7 | Increased | Sf3b1 | Increased |
| Slc16a9 | Increased | Rack1 | Increased | Srprb | Increased |
| Ifit1 | Increased | Rap1b | Increased | Srsf3 | Increased |
| Oas3 | Increased | Rbp2 | Increased | Tial1 | Increased |

TABLE 4

| Biomarker | Expression |
|---|---|
| ASCL2 | Increased |
| CLDN4 | Increased |
| IGFBP4 | Increased |
| SOX4 | Increased |
| CDC42EP1 | Increased |
| ZNF358 | Increased |
| SP5 | Increased |
| MUC6 | Increased |
| ANXA10 | Increased |
| PGC | Increased |
| MAL | Increased |
| IL1RN | Increased |
| ANXA8L1 | Increased |
| SLFN12L | Increased |
| SLFN12 | Increased |
| CYP3A43 | Increased |
| NXF2 | Increased |
| NXF2B | Increased |
| IFIT1B | Increased |
| CYP2C8 | Increased |
| SLC5A4 | Increased |
| SLC6A1 | Increased |
| ITGA7 | Increased |
| KHDC1 | Increased |
| GK3P | Increased |
| STC2 | Increased |
| AN01 | Increased |
| SPRR2G | Increased |
| SPP1 | Increased |
| ABCA12 | Increased |
| ANXA8 | Increased |
| EPB41L1 | Increased |
| SLC26A9 | Increased |
| NRP2 | Increased |
| GKN1 | Decreased |
| PRNP | Decreased |
| CAS3 | Decreased |
| AHNAK | Decreased |
| DDX60 | Decreased |
| DST | Decreased |
| GK | Decreased |

TABLE 5

| Biomarker | Expression |
|---|---|
| CDKN2B | Increased |
| UGT2A3 | Increased |
| DHRS9 | Increased |
| FABP1 | Increased |
| TMIGD1 | Increased |
| KRT20 | Increased |
| SLC6A19 | Increased |
| PDZK1 | Increased |
| PLB1 | Increased |
| CREB3L3 | Increased |
| SLC15A1 | Increased |
| XPNPEP2 | Increased |
| NAALADL1 | Increased |
| SLC51A | Increased |
| GDPD2 | Increased |
| ACE2 | Increased |
| FMO5 | Increased |
| MOGAT2 | Increased |
| PLEKHG6 | Increased |
| MAPRE3 | Increased |
| SGK2 | Increased |
| CLIC5 | Increased |
| IGSF9 | Increased |
| CYP4F2 | Increased |
| CIDEC | Increased |
| PDLIM2 | Increased |
| TREH | Increased |
| G6PC | Increased |
| SLC26A6 | Increased |
| SLC23A1 | Increased |
| PTPRH | Increased |
| MAF | Increased |
| GDA | Increased |
| AKR1B10 | Increased |
| CD36 | Increased |
| EMP1 | Increased |
| FAM78A | Increased |
| LHFPL2 | Increased |
| VWA1 | Increased |
| AREG | Decreased |
| CFI | Decreased |
| APLP1 | Increased |
| SLC47A1 | Increased |
| ASPA | Increased |
| BGLAP | Increased |
| ADRB2 | Decreased |
| PLK2 | Decreased |
| KLF11 | Decreased |
| EGR4 | Decreased |
| FAM71A | Decreased |
| PTPRM | Decreased |
| GPR3 | Decreased |
| PLK3 | Decreased |
| PODN | Increased |

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments or aspects presented herein.

FIG. 1. Schematic outline of the experimental design. Lgr5^{EGFP-IRES-creERT2} mice were crossed with Villin-Cre;Msh2^{LoxP/LoxP} mice (VC-Msh2^{LoxP/LoxP}). After crypt isolation from Msh2-WT, Msh2-HET, or Msh2-KO mice, FACS was performed to isolate GFP labeled Lgr5^{EGFP+} stem cells or Lgr5^{EGFP-} daughter cells. Sorted cell populations were used to extract RNA and protein for transcriptomics and proteomics profiling by mRNAseq and tandem Mass Spectrometry, respectively. Bioinformatic analyses were used to identify differentially expressed genes and proteins in MMRd and haploinsufficient ISC. Validation of gene expression signatures was performed using both mouse tissue specimens and organoids as well as human cell lines and tissues from LS patients.

FIGS. 2A-D. Bioinformatics analysis of gene expression from Lgr5^{EGFP+} stem and Lgr5^{EGFP-} daughter cells. (A) PCA plot of expression profiles from Lgr5^{EGFP+} intestinal stem cells (solid colors) isolated from Msh2-WT (dark yellow), Msh2-HET (dark blue) and Msh2-KO (dark red) and from daughter cells (Lgr5^{EGFP-} and EpCAM+, light shades of respective colors). The first and second principal components are plotted in the X and Y-axis, respectively. Individual samples within each group are connected by a centroid. A total of 21 mice for Msh2-WT, 25 for Msh2-HET and 40 for Msh2-KO were equally distributed among three biological replicates to obtain ˜10,000 ISC for each replicate per genotype; (B) Volcano plots illustrate genes expressed in ISC of Msh2-KO and Msh2-HET compared to Msh2-WT and in ISC of Msh2-KO as compared to Msh2-HET. X-axis presents Log2Fold change and Y-axis presents log10 of adjusted P-value for multiple comparisons from DESeq2 differential analysis. The horizontal dashed line represents FDR=0.05, while left and right vertical dashed lines represent Log2FC of ±1, respectively. Significantly down-regulated genes are displayed in green, upregulated in red, and non-significant in black; (C) Venn diagrams showing numbers of significantly expressed genes in Lgr5^{EGFP+} stem cells and Lgr5^{EGFP-} non-stem cells for each genotype compared with Msh2-WT; (D) Validation of the expression of key signature genes from the MMRd and MMR-haploinsufficient signatures as well as stem and differentiation markers analyzed using qRT-PCR in FACS sorted Lgr5^{EGFP+} (stem, left panels) and Lgr5^{EGFP-} (non-stem, right panels) cells from Msh2-WT, Msh2-HET and Msh2-KO. Data is presented as fold changes and depicted as relative gene expression levels compared to expression levels in Lgr5^{EGFP+} cells of Msh2-WT as reference. Expression levels of Gapdh were used as an internal housekeeping gene for normalization. Error bars display ±SD. One-way ANOVA with Tukey's multiple comparison post-hoc test, *P -value<0.05, **P-value <0.01, ***P-value<0.001, ****P-value<0.0001.

FIGS. 3A-C. Bubble chart plots display statistically significant pathways enriched in Msh2-KO ISCs (A), Msh2-HET ISCs (B), and both Msh2-HET and Msh2-KO ISCs (C), using BH-adjusted P-value=0.05 as a cutoff. Pathways bolded were relevant in terms of function to the molecular biology of MMRd ISC. The size of circles represents adjusted P-value (larger circles represent smaller P-value). The colors of bubbles were determined by the sign and amplitude of normalized enrichment score (NES) with positively enriched pathways in red and negatively enriched pathways in green.

FIGS. 4A-B. Expression of Msh2-HET and Msh2-KO signatures in FAP and LS patient samples. (A) Unsupervised hierarchical clustering heatmaps showing the expression pattern of selected Msh2-KO versus Msh2-WT signature genes in FAP polyps and LS hypermutant/MSI pre-cancer/tumor samples (FDR<0.05 in human and same fold change direction in both mouse and human); (B) Expression patterns of selected Msh2-KO versus Msh2-HET signature genes in normal mucosa from LS patients using row-centered and batch corrected expression data (FDR<0.05 in human and same fold change direction in mouse and human) Dendrograms indicate sample-sample Pearson correlation distances. The significance of genes in human comparison are indicated in a row covariate bar. Log2FC in human and mice for each gene are shown as scatter plots. Sample type is color-coded as follows: blue represents normal tissue from LS patients; red represents hypermutant/MSI adenomas/tumor tissue from LS patients, respectively; FC, fold change; LS, Lynch Syndrome.

FIGS. 5A-C. Expression and localization of Spp1 within crypts of MMR mouse models and LS patient specimens. (A) Small intestine from 8 week-old Msh2-WT, Msh2-HET, and Msh2-KO mice was stained with antibodies against GFP to detect Lgr5⁺ cells (green) and Spp1 (red) by immunofluorescence. Panels show representative images for Lgr5 and Spp1 expression and location within crypts. Yellow arrows in merged images demarcate the co-localization of Lgr5 and Spp1 in crypts of tissues from Msh2-HET and Msh2-KO. Nuclei were counterstained with DAPI (blue). Scale bar is equivalent to 50 μm; (B) Representative images from FFPE tissue sections of H&E, nuclear counterstaining with DAPI (blue), immunostaining with anti-human LGR5 (TSA with Opal-520, green), and anti-SPP1 (TSA with Opal-570, red) antibodies, and composite images acquired using fluorescent multiplex immunohistochemistry. Regions of interest for digital image analysis include normal colon epithelium (top panel), adenomas (middle panel), and adenocarcinoma (lower panel) displaying single positive cells for LGR5 and SPP1, and double-positive cells (MERGE); Scale bar represents 100 μm and scale bar in insets are equivalent to 10 μm. (C) The number of positive cells for each marker and double positives reported was quantified as cell density and expressed by the number of cells per mm² using inForm advance image analysis software (considering that the total number of nucleated cells is 100%). The percentage of stem cells co-expressing SPP1 and LGR5 (double-positive cells) was higher in pre-cancers and cancers compared to normal adjacent tissue showing a non-statistically significant trend. Graph displays mean±SEM.

FIGS. 6A-D. Isolation of Lgr5^{EGFP+} ISCs and Lgr5^{EGFP-} daughter cells using fluorescence-activated cell sorting (FACS). Crypt preparations from each genotype were sorted after calibrating FACS equipment with crypts prepared from non-GFP expressing mice (A). First, cells were stained with CD45 antibody, and those CD45− cells were sorted out to eliminate lymphocytes (marked by an arrow). Then, the inventors incubated cells with EpCAM antibody and gated cells for EpCAM+ or GFP− high after duly calibrating and adjusting the GFP gates using non-GFP cells from non-GFP expressing mice and EpCAM+ cells. Numbers shown in the FACS gates are the recovery percentage of Lgr5^{EGFP+} cells from Msh2-WT (B), Msh2-HET (C), and Msh2-KO (D) mice.

FIG. 7. Recovery of Lgr5EGFP+ stem cells by FACS. Average recovery of Lgr5EGFP+ cells obtained from crypt preparations of small intestines of Msh2-WT, Msh2-HET, and Msh2-KO mice. Mean recovery of Lgr5EGFP+ cells from Msh2-WT mice was 224,388 (n=27), 47,873 from Msh2-HET mice (n=35), and 10,392 from Msh2-KO mice (n=57). Error bars represent ±SD. One-way ANOVA with Tukey's multiple comparison post-hoc test, **P-value<0.01, ****P-value<0.0001.

FIGS. 8A-E. Transcriptomic analysis for differentially expressed genes (DEGs) from Lgr5^{EGFP-} daughter cells derived from crypts of Msh2 haplo-insufficient and MMR deficient mice. (A) PCA plot of expression profiles from Lgr5^{EGFP+} intestinal stem cells (solid colors, left panel) isolated from Msh2-WT (yellow), Msh2-HET (blue), and Msh2-KO (red) mice and from daughter cells (Lgr5^{EGFP-} and EpCAM+, light shades of respective colors, right panel). The first and second principal components are plotted as X-axis and Y-axis, respectively; (B) Volcano plots illustrating unique genes expressed in daughter cell fractions (Lgr5EGFP−) of either Msh2-HET or Msh2-KO as compared to Lgr5^{EGFP-} daughter cells from Msh2-WT mice. Volcano plot for genes significantly dysregulated in Lgr5EGFP− cells of Msh2-KO as compared to Lgr5^{EGFP-} Msh2-HET cells is also shown. X-axis: Log2 FC (fold change); Y-axis: log10 of BH adjusted P-value from DESeq2 differential analysis. Significantly dysregulated genes are annotated with downregulated genes in green (FDR≤0.05 and Log2FC≤−1), up-regulated genes in red (FDR≤0.05 and Log2FC≥1), and non-significant genes in black. The top 5 up-regulated genes are indicated with arrows; (C) Numbers of differentially expressed genes and the intersection between stem cell and non-stem cell comparisons. UpSet plot showing numbers of differentially expressed genes (DEGs) at FDR≤0.05 and |Log2FC|≥1 identified in each genotype and corresponding stem cells (Lgr5^{EGFP+}) and daughter (Lgr5^{EGFP-}) cell fractions, as well as intersection sets between comparisons. Colors represent different levels of intersections, with light blue representing the number of significant genes in a single comparison, while red, light green, and dark green representing intersection size among two, three, and four comparisons, respectively. The comparisons are highlighted as dots and connected with lines in corresponding colors. The top bar plots (Intersection size) are the numbers of significant common genes for annotated intersections, while the right bar plots (Set size) are the number of significant genes for each corresponding comparison. Venn diagram showing ‘at a glance’ number of unique intersecting genes between Lgr5^{EGFP+} ISC fractions of different genotypes and numbers in parenthesis indicate the number of differentially expressed genes for a particular genotype at a given significance level and fold change; (D) Freshly isolated small intestines from Msh2-WT and Msh2-KO mice were used for organoid preparation. Macrophage Colony Stimulating Factor 1 (M-CSF1) was added to the CMGF++ media at a concentration of 50 ng/mL (in PBS) for 24 hours. Gene expression was performed using SYBR Green chemistry (ThermoFisher) and analyzed by ΔΔCt methodology. All PCR experiments were performed in triplicate. Results are shown as fold change in gene expression; (E) Numbers of differentially expressed genes and the intersection between MMRd and MMRd (from reference 17) stem cell comparisons. UpSet plot showing numbers of differentially expressed genes (DEGs) at FDR≤0.05 and |Log2FC|≥1 identified in each genotype and corresponding stem cells (Lgr5^{EGFP+}) cell fractions, as well as intersection sets between comparisons. Colors represent different levels of intersections, with red representing the number of significant genes in a single comparison, while blue, light green, and dark green representing intersection size among two, three, and four comparisons, respectively. The comparisons are highlighted as dots and connected with lines in corresponding colors. The top bar plots (Intersection size) are the numbers of significant common genes for annotated intersections, while the right bar plots (Set size) are the number of significant genes for each corresponding comparison.

FIGS. 9A-C. Overlapping of unique signature genes in transcriptomic (RNAseq) and proteomic profile of MMR deficiency and functional network of differentially expressed proteins. (A) Heatmaps are shown for 27 proteins that overlap with MMR deficient (Msh2-KO/Lgr5^{EGFP+}) gene signature. Expression of each replicate used for RNAseq and proteomic expression intensities as spectral counts are displayed as a heatmap. Expression of genes in RNAseq and spectral counts for protein intensity from Lgr5^{EGFP+} stem cells of Msh2-KO were compared with expression profiles observed in similar fractions of Msh2-WT and also with Lgr5^{EGFP-} fractions of Msh2-KO. Missing values for any replicate in proteomic spectral counts are shown in black and centered at spectral count of zero; (B) The top scoring of protein interaction networks ‘Cellular development, cellular growth and proliferation, Hematological System Development and Function’ depicted for Msh2-KO/Lgr5^{EGFP+}; (C) ‘Cell Cycle, DNA replication, Recombination and Repair, RNA post-transcriptional modification’ for Msh2-HET/Lgr5^{EGFP+} generated through Ingenuity Pathway Analysis (IPA) (Qiagen Inc.). The shapes represent the molecular class of proteins in the network. Solid and dashed lines indicate direct and indirect interactions, respectively.

FIGS. 10A-C. Transcriptomic analysis of the Msh2-KO and Msh2-HET signatures in human organoids and tissue samples. (A) Expression of Msh2-HET signature in FAP and LS patient samples. Unsupervised hierarchical clustering heatmap showing the expression pattern of selected Msh2-HET versus Msh2-WT signature genes (same fold change direction in mouse and human) in normal mucosa from LS and FAP patients using row-centered and batch corrected expression data. Dendrograms indicate sample-sample Pearson correlation distances. The significant genes in human comparison are indicated in a row covariate bar. Log2FC in human and corresponding mice comparisons are shown as scatter plots. The sample type is color-coded as follows: blue represents normal tissue from LS patients; yellow represents normal mucosa from FAP patients (for Msh2-KO signature validation), respectively; FC, fold change; FAP, familial adenomatous polyposis; LS, Lynch Syndrome; (B) Validation of the combined Msh2-HET and Msh2-KO signatures in human organoids from publicly available data sets. Expression patterns of genes with FDR≤0.05 and log2FoldChange≥1.5 were displayed in an unsupervised hierarchical clustering heatmap generated using Bioconductor R package ComplexHeatmap. Normal mucosa organoids from LS patients (22) (LS Organoids) are displayed in cyan and normal mucosa organoids from sporadic CRC patients (23) (Sporadic Organoids) are displayed in pink; (C) Chromatin immunoprecipitation (ChIP) assays were performed in chromatin extracts from the MMRp colorectal cancer cell line SW620 and the MMRd endometrial cancer cell line HEC59, which harbors bi-allelic inactivating MSH2 mutations, thus resembling the LS mouse model. The inventors observed that the expression of the SPP1 is significantly up-regulated in the MMRd cell line HEC59 compared to the MMRp cell line SW620 (left panel), which is consistent with the Sppl upregulation observed in the intestinal stem cells of Msh2-KO mice compared to Msh2-WT. In addition, the inventors observed that the levels of the repressive histone mark H3K27me3 were much higher in the promoter region compared to the 3′UTR region in the MMRp SW620 cell line. Furthermore, the levels of H3K4me3, which is known to be localized near the proximal promoter of the actively transcribed gene, were within the background levels. These data suggest that H3K27methylation at the SPP1 gene caused strong repression in MMRp cells. On the other hand, the levels of H3K4me3 were much higher at the SPP1 promoter in HEC59 cells compared to the control 3′UTR region, thus suggesting that the presence of H3K4me3 in the promoter region correlates with active gene transcription. Enrichment of chromatin fragments by control IgG antibody was almost negligible, thus suggesting the specificity of H3K4me3 and H3K27me3 antibodies for ChIP assays. All experiments were performed in triplicates.

FIG. 11. Automated quantitative imaging of fluorescent multiplex immunohistochemistry. Representative images from FFPE tissue sections stained with anti-human LGR5 (TSA with Opal-520, green), SPP1 (TSA with Opal-570, red) antibodies, nuclear counterstaining with DAPI (blue), and the composite image corresponding to FIG. 5B. Regions of interest for digital image analysis included normal colon epithelium (top panel), adenoma (middle panel), and adenocarcinoma (lower panel).

FIG. 12 shows cytoplasmic staining of Spp1 by IHC and the H-score for subjects having normal, tubular adenoma, or adenocarcinoma tissues.

### DETAILED DESCRIPTION

Lynch Syndrome (LS) is the most common cause of hereditary colorectal cancer (CRC) and is secondary to germline alterations in one of four DNA mismatch repair (MMR) genes. The inventors sought to provide novel insights into the initiation of MMRd colorectal carcinogenesis by characterizing the expression profile of MMR deficient (MMRd) intestinal stem cells (ISC). A tissue-specific MMRd mouse model (Villin-Cre;Msh2LoxP/LoxP) was crossed with a reporter mouse (Lgr5-EGFP-IRES-creERT2) to trace and isolate ISCs (Lgr5+) using flow cytometry. Three different ISC genotypes (Msh2-KO, Msh2-HET, and Msh2-WT) were isolated, and mRNAseq and mass spectrometry was performed, followed by bioinformatic analyses to identify expression signatures of complete MMRd and haplo-insufficiency. Then, the inventors validated these findings using qRT-PCR, immunohistochemistry (IHC), and whole transcriptomic sequencing in mouse tissues, organoids, and a cohort of human normal colorectal mucosa, pre-cancers, and early-stage CRC from LS and Familial Adenomatous Polyposis (FAP) patients as controls. The inventors observed that Msh2-KO ISC clustered together with differentiated intestinal epithelial cells from all genotypes. Gene set enrichment analysis indicated inhibition of replication, cell cycle progression, and the Wnt pathway, and activation of epithelial signaling, and immune reaction. The expression signature of MMRd was able to distinguish neoplastic lesions between LS patients and FAP controls. It was observed that SPP1 was specifically upregulated in MMRd ISC and observed colocalization with LGR5 in colorectal LS pre-cancers and tumors. Overall, expression signatures of MMRd ISC recapitulate the initial steps of LS carcinogenesis and have the potential to unveil novel biomarkers of early initiation of carcinogenesis.

### I. DEFINITIONS

“Prognosis” refers to as a prediction of how a patient will progress, and whether there is a chance of recovery. “Cancer prognosis” generally refers to a forecast or prediction of the probable course or outcome of the cancer, with or without a treatment. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis also includes prediction of favorable responses to cancer treatments, such as a conventional cancer therapy. A response may be either a therapeutic response (sensitivity or recurrence-free survival) or a lack of therapeutic response (residual disease, which may indicate resistance or recurrence).

The terms “substantially the same,” “not significantly different, ” or “within the range” refers to a level of expression that is not significantly different than what it is compared to. Alternatively, or in conjunction, the terms refer to a level of expression that is less than 2, 1.5, or 1.25 fold different or less than 2, 1, or 0.5 standard deviations than the expression or activity level it is compared to.

By “subject” or “patient” is meant any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

The term “primer” or “probe” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. A probe may also refer to a nucleic acid that is capable of hybridizing by base complementarity to a nucleic acid of a gene of interest or a fragment thereof.

As used herein, “increased expression” or “elevated expression” or “decreased expression” refers to an expression level of a biomarker in the subject's sample as compared to a reference level representing the same biomarker or a different biomarker. In certain aspects, the reference level may be a reference level of expression from a non-cancerous tissue from the same subject. Alternatively, the reference level may be a reference level of expression from a different subject or group of subjects. For example, the reference level of expression may be an expression level obtained from a sample (e.g., a tissue, fluid or cell sample) of a subject or group of subjects without cancer, with fast doubling time HCC, or with slow doubling time HCC, or an expression level obtained from a non-cancerous tissue of a subject or group of subjects with cancer. The reference level may be a single value or may be a range of values. The reference level of expression can be determined using any method known to those of ordinary skill in the art. The reference level may also be depicted graphically as an area on a graph. In certain aspects, a reference level is a normalized level.

The term “determining” or “evaluating” as used herein may refer to measuring, quantitating, or quantifying (either qualitatively or quantitatively).

### II. COLORECTAL CANCER STAGING AND TREATMENTS

Methods and compositions may be provided for treating colorectal cancer with particular applications of biomarker expression or activity levels. Based on a profile of biomarker expression or activity levels, different treatments may be prescribed or recommended for different cancer patients.

A. Cancer staging

Colorectal cancer, also known as colon cancer, rectal cancer, or bowel cancer, is a cancer from uncontrolled cell growth in the colon or rectum (parts of the large intestine), or in the appendix. Certain aspects of the methods are provided for patients that are stage I-IV colorectal cancer patients. In particular aspects, the patient is a stage II or III patient. In a further aspect, the patient is a stage I or II patient. In a further aspect, the patient is a stage I, II, or III patient.

The most common staging system is the TNM (for tumors/nodes/metastases) system, from the American Joint Committee on Cancer (AJCC). The TNM system assigns a number based on three categories. “T” denotes the degree of invasion of the intestinal wall, “N” the degree of lymphatic node involvement, and “M” the degree of metastasis. The broader stage of a cancer is usually quoted as a number I, II, III, IV derived from the TNM value grouped by prognosis; a higher number indicates a more advanced cancer and likely a worse outcome. Details of this system are in the graph below:

| AJCC | | TNM stage criteria |
|---|---|---|
| stage | TNM stage | for colorectal cancer |
| Stage 0 | Tis N0 M0 | Tis: Tumor confined to mucosa; |
| | | cancer-in-situ |
| Stage I | T1 N0 M0 | T1: Tumor invades submucosa |
| Stage I | T2 N0 M0 | T2: Tumor invades muscularis propria |
| Stage II-A | T3 N0 M0 | T3: Tumor invades subserosa or beyond |
| | | (without other organs involved) |
| Stage II-B | T4 N0 M0 | T4: Tumor invades adjacent organs or |
| | | perforates the visceral peritoneum |
| Stage III-A | T1-2 N1 M0 | N1: Metastasis to 1 to 3 regional lymph |
| | | nodes. T1 or T2. |
| Stage III-B | T3-4 N1 M0 | N1: Metastasis to 1 to 3 regional lymph |
| | | nodes. T3 or T4. |
| Stage III-C | any T, N2 | N2: Metastasis to 4 or more regional lymph |
| | M0 | nodes. Any T. |
| Stage IV | any T, any | M1: Distant metastases present. |
| | N, M1 | Any T, any N. |

### B. Therapy

For people with localized and/or early colorectal cancer, the preferred treatment is complete surgical removal with adequate margins, with the attempt of achieving a cure. This can either be done by an open laparotomy or sometimes laparoscopically. Sometimes chemotherapy is used before surgery to shrink the cancer before attempting to remove it (neoadjuvant therapy). The two most common sites of recurrence of colorectal cancer is in the liver and lungs. In some aspects, the treatment of early colorectal cancer excludes chemotherapy. In further aspects, the treatment of early colorectal cancer includes neoadjuvant therapy (chemotherapy or radiotherapy before the surgical removal of the primary tumor), but excludes adjuvant therapy (chemotherapy and/or radiotherapy after surgical removal of the primary tumor.

In both cancer of the colon and rectum, chemotherapy may be used in addition to surgery in certain cases. In rectal cancer, chemotherapy may be used in the neoadjuvant setting.

In certain aspects, there may be a decision regarding the therapeutic treatment based on biomarker expression. Chemotherapy based on antimetabolites or thymidylate synthase inhibitors such as fluorouracil (5-FU) have been the main treatment for metastatic colorectal cancer. Major progress has been made by the introduction of regimens containing new cytotoxic drugs, such as irinotecan or oxaliplatin. The combinations commonly used, e.g., irinotecan, fluorouracil, and Jeucovorin (FOLFIRI) and oxaliplatin, fluorouracil, and leucovorin (FOLFOX) can reach an objective response rate of about 50% . However, these new combinations remain inactive in one half of the patients and, in addition, resistance to treatment appear in almost all patients who were initially responders. More recently, two monoclonal antibodies targeting vascular endothelial growth factor Avastin® (bevacizumab) (Genentech Inc., South San Francisco CA) and epidermal growth factor receptor Erbitux®(cetuximab) (Imclone Inc. New York City) have been approved for treatment of metastatic colorectal cancer but are always used in combination with standard chemotherapy regimens. In some aspects, the cancer therapy may include one or more of the chemical therapeutic agents including thymidylate synthase inhibitors or antimetabolites such as fluorouracil (5-FU), alone or in combination with other therapeutic agents.

For example, in some aspects, the first treatment to be tested for response therapy may be antimetabolites or thymidylate synthase inhibitors, prodrugs, or salts thereof. In some aspects, this treatment regimen is for advanced cancer. In some aspects, this treatment regimen is excluded for early cancer.

Antimetabolites can be used in cancer treatment, as they interfere with DNA production and therefore cell division and the growth of tumors. Because cancer cells spend more time dividing than other cells, inhibiting cell division harms tumor cells more than other cells. Anti-metabolites masquerade as a purine (azathioprine, mercaptopurine) or a pyrimidine, chemicals that become the building-blocks of DNA. They prevent these substances becoming incorporated in to DNA during the S phase (of the cell cycle), stopping normal development and division. They also affect RNA synthesis. However, because thymidine is used in DNA but not in RNA (where uracil is used instead), inhibition of thymidine synthesis via thymidylate synthase selectively inhibits DNA synthesis over RNA synthesis. Due to their efficiency, these drugs are the most widely used cytostatics. In the ATC system, they are classified under LO1B. In some aspects, this treatment regimen is for advanced cancer. In some aspects, this treatment regimen is excluded for early cancer.

Thymidylate synthase inhibitors are chemical agents which inhibit the enzyme thymidylate synthase and have potential as an anticancer chemotherapy. As an anti-cancer chemotherapy target, thymidylate synthetase can be inhibited by the thymidylate synthase inhibitors such as fluorinated pyrimidine fluorouracil, or certain folate analogues, the most notable one being raltitrexed (trade name Tomudex). Five agents were in clinical trials in 2002: raltitrexed, pemetrexed, nolatrexed, ZD9331, and GS7904L. Additional non-limiting examples include: Raltitrexed, used for colorectal cancer since 1998; Fluorouracil, used for colorectal cancer; BGC 945; OSI-7904L. In some aspects, this treatment regimen is for advanced cancer. In some aspects, this treatment regimen is excluded for early cancer.

In further aspects, there may be involved prodrugs that can be converted to thymidylate synthase inhibitors in the body, such as Capecitabine (INN), an orally-administered chemotherapeutic agent used in the treatment of numerous cancers. Capecitabine is a prodrug, that is enzymatically converted to 5-fluorouracil in the body. In some aspects, this treatment regimen is for advanced cancer. In some aspects, this treatment regimen is excluded for early cancer.

If cancer has entered the lymph nodes, adding the chemotherapy agents fluorouracil or capecitabine increases life expectancy. If the lymph nodes do not contain cancer, the benefits of chemotherapy are controversial. If the cancer is widely metastatic or unresectable, treatment is then palliative. For example, a number of different chemotherapy medications may be used. Chemotherapy agents for this condition may include capecitabine, fluorouracil, irinotecan, leucovorin, oxaliplatin and UFT. Another type of agent that is sometimes used are the epidermal growth factor receptor inhibitors. In some aspects, this treatment regimen is for advanced cancer. In some aspects, this treatment regimen is excluded for early cancer.

In certain aspects, alternative treatments may be prescribed or recommended based on the biomarker profile. In addition to traditional chemotherapy for colorectal cancer patients, cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing. In some aspects, treatment with one or more of the compounds described herein is for advanced cancer. In some aspects, treatment with one or more of the compounds described herein is excluded for early cancer.

While a combination of radiation and chemotherapy may be useful for rectal cancer, its use in colon cancer is not routine due to the sensitivity of the bowels to radiation. Just as for chemotherapy, radiotherapy can be used in the neoadjuvant and adjuvant setting for some stages of rectal cancer. In some aspects, this treatment regimen is for advanced cancer. In some aspects, this treatment regimen is excluded for early cancer.

In people with incurable colorectal cancer, treatment options including palliative care can be considered for improving quality of life. Surgical options may include non-curative surgical removal of some of the cancer tissue, bypassing part of the intestines, or stent placement. These procedures can be considered to improve symptoms and reduce complications such as bleeding from the tumor, abdominal pain and intestinal obstruction. Non-operative methods of symptomatic treatment include radiation therapy to decrease tumor size as well as pain medications. In some aspects, this treatment regimen is for advanced cancer. In some aspects, this treatment regimen is excluded for early cancer.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. In some aspects, this treatment regimen is for advanced cancer. In some aspects, this treatment regimen is excluded for early cancer.

Generally, the tumor cell must bear some marker that is amenable to targeting, i.e., is not present on the majority of other cells. Many tumor markers exist and any of these may be suitable for targeting. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. Markers described herein may be used in the context of the current claims for the purposes of developing a targeting moiety. For example, the targeting moiety may be one that binds the tumor marker. In some aspects, the targeting moiety is an antibody. In further aspects, the targeting moiety is an aptamer or aptamir.

In yet another aspect, the treatment is a gene therapy. In certain aspects, the therapeutic gene is a tumor suppressor gene. A tumor suppressor gene is a gene that, when present in a cell, reduces the tumorigenicity, malignancy, or hyperproliferative phenotype of the cell. This definition includes both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell. Examples of tumor suppressor nucleic acids within this definition include, but are not limited to APC, CYLD, HIN-I, KRAS2b, pló, p19, p21, p2′7, p27mt, p53, p5′7, p′73, PTEN, Rb, Uteroglobin, Skp2, BRCA-I, BRCA-2, CHK2, CDKN2A, DCC, DPC4, MADR2/JV18, MEN1, MEN2, MTS1, NF1, NF2, VHL, WRN, WT1, CFTR, C-CAM, CTS-I, zacl, scFV, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes at www.cise.ufl.edu/-yyl/HTML-TSGDB/Homepage.litml. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied.

### C. Monitoring

In certain aspects, the biomarker-based method may be combined with one or more other colon cancer diagnosis or screening tests at increased frequency if the patient is determined to be at high risk for recurrence or have a poor prognosis based on the biomarker described above.

The colon monitoring may include any methods known in the art. In particular, the monitoring include obtaining a sample and testing the sample for diagnosis. For example, the colon monitoring may include colonoscopy or coloscopy, which is the endoscopic examination of the large bowel and the distal part of the small bowel with a CCD camera or a fiber optic camera on a flexible tube passed through the anus. It can provide a visual diagnosis (e.g. ulceration, polyps) and grants the opportunity for biopsy or removal of suspected colorectal cancer lesions. Thus, colonoscopy or coloscopy can be used for treatment.

In further aspects, the monitoring diagnosis may include sigmoidoscopy, which is similar to colonoscopy—the difference being related to which parts of the colon each can examine A colonoscopy allows an examination of the entire colon (1200-1500 mm in length). A sigmoidoscopy allows an examination of the distal portion (about 600 mm) of the colon, which may be sufficient because benefits to cancer survival of colonoscopy have been limited to the detection of lesions in the distal portion of the colon. A sigmoidoscopy is often used as a screening procedure for a full colonoscopy, often done in conjunction with a fecal occult blood test (FOBT). About 5% of these screened patients are referred to colonoscopy.

In additional aspects, the monitoring diagnosis may include virtual colonoscopy, which uses 2D and 3D imagery reconstructed from computed tomography (CT) scans or from nuclear magnetic resonance (MR) scans, as a totally non-invasive medical test.

The monitoring include the use of one or more screening tests for colon cancer including, but not limited to fecal occult blood testing, flexible sigmoidoscopy and colonoscopy. Of the three, only sigmoidoscopy cannot screen the right side of the colon where 42% of malignancies are found. Virtual colonoscopy via a CT scan appears as good as standard colonoscopy for detecting cancers and large adenomas but is expensive, associated with radiation exposure, and cannot remove any detected abnormal growths like standard colonoscopy can. Fecal occult blood testing (FOBT) of the stool is typically recommended every two years and can be either guaiac based or immunochemical. Annual FOBT screening results in a 16% relative risk reduction in colorectal cancer mortality, but no difference in all-cause mortality. The M2-PK test identifies an enzyme in colorectal cancers and polyps rather than blood in the stool. It does not require any special preparation prior to testing. M2-PK is sensitive for colorectal cancer and polyps and is able to detect bleeding and non-bleeding colorectal cancer and polyps. In the event of a positive result people would be asked to undergo further examination e.g. colonoscopy.

### III. ROC ANALYSIS

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1-specificity). The ROC curve is thus the sensitivity as a function of fall-out. In general, if the probability distributions for both detection and false alarm are known, the ROC curve can be generated by plotting the cumulative distribution function (area under the probability distribution from —infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz CE (1978) Basic principles of ROC analysis. Seminars in Nuclear Medicine 8:283-298; Youden WJ (1950) An index for rating diagnostic tests. Cancer 3:32-35; Zweig MH, Campbell G (1993) Receiver-operating characteristic (ROC) plots: a fundamental evaluation tool in clinical medicine. Clinical Chemistry 39:561-577; and Greiner M, Pfeiffer D, Smith RD (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### IV. SAMPLE PREPARATION

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from intestinal or mucosal tissue by any of the biopsy methods previously mentioned. In other aspects the sample may be obtained from any of the tissues provided herein that include but are not limited to non-cancerous or cancerous tissue and non-cancerous or cancerous tissue from the mucosa, colon mucosa, serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor, polyp, or neoplasm. In yet other aspects the polyp, tumor, or neoplasm is in the colon or in colon tissues. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple colon tissue samples may be obtained for diagnosis by the methods described herein. In other cases, multiple samples, such as one or more samples from one tissue type (for example colon) and one or more samples from another specimen (for example serum) may be obtained for diagnosis by the methods. In some cases, multiple samples such as one or more samples from one tissue type (e.g. colon) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, colonoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. NUCLEIC ACID ASSAYS

Aspects of the methods include assaying nucleic acids to determine expression or activity levels. Arrays can be used to detect differences between two samples. Specifically contemplated applications include identifying and/or quantifying differences between RNA from a sample that is normal and from a sample that is not normal, between a cancerous condition and a non-cancerous condition, between one cancerous condition, such as fast doubling time cells and another cancer condition, such as slow doubling time cells, or between two differently treated samples. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789, 162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HP A)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNASeq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### VI. PROTEIN ASSAYS

A variety of techniques can be employed to measure expression levels of polypeptides and proteins in a biological sample. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et at, Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### VII. ADMINISTRATION OF THERAPEUTIC COMPOSITIONS

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VIII. PHARMACEUTICAL COMPOSITIONS

In certain aspects, the compositions or agents for use in the methods, such as chemotherapeutic agents or biomarker modulators, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### IX. KITS

Certain aspects of the present invention also concern kits containing compositions of the invention or compositions to implement methods of the invention. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some aspects, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, primers, synthetic nucleic acids, nonsynthetic nucleic acids, biomarker binding polypeptides, antibodies, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker, and any such molecules that hybridize to a biomarker nucleic acid.

Aspects of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample and/or probes and detecting agents. The kit may also include labeling reagents, including at least one of amine-modified nucleotide, poly(A) polymerase, and poly(A) polymerase buffer. Labeling reagents can include an amine-reactive dye.

It is contemplated that any method or composition described herein can be implemented with respect to any other method or composition described herein and that different aspects may be combined. The claims originally filed are contemplated to cover claims that are multiply dependent on any filed claim or combination of filed claims.

Any aspect of the disclosure involving a specific biomarker is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—The Transcriptomic Landscape of Mismatch Repair-Deficient Intestinal Stem Cells

### A. Introduction

Lynch Syndrome (OMIM# 120435, LS) is a hereditary cancer syndrome predisposing patients to develop colorectal cancers (CRC) as well as tumors of the endometrium, ovary, stomach, and small intestine (1). LS has an estimated prevalence of 1 in 279, thus affecting over 1 million individuals in the US (2). LS is secondary to germline mutations in one of the DNA mismatch repair (MMR) genes (MLH1, MSH2, MSH6, and PMS2) that control post-replicative DNA proofreading, thus ensuring genomic integrity (3). MMR deficiency (MMRd) accelerates the acquisition of secondary somatic mutations in oncogenes and tumor suppressor genes that regulate different pathways, including cell fate, transcription, growth factors, and other DNA repair mechanisms, thus promoting carcinogenesis (4). LS has an autosomal dominant inheritance causing an estimated lifetime risk of CRC development of 20-50% depending on the germline MMR gene that carries the mutation, and also a young age of onset, typically in the fourth decade of life (5-7). Despite the recommendation of annual or biannual endoscopic surveillance starting at age 20-25, LS patients continue developing interval cancers and are counseled to consider risk-reducing surgeries (8, 9).

The epithelium of the small and large intestine contains niches of stem cells located at the bottom of specialized finger-like invaginations that are arranged in functional units called crypts, which are surrounded by connective tissue, and the underlying lamina propria. These fast-cycling stem cells, refered to as crypt base columnar (CBC) cells, generate daughter cells that exit the stem cell niche to integrate into the transit-amplifying compartment by migrating upwards to the lumen of the gut. This process takes 4-5 days and gives rise to several differentiated and specialized cell subtypes, including enterocytes (nutrient uptake), goblet (mucus production), enteroendocrine (hormone production), and paneth cells (growth factor production exclusively in the small intestine) (11-13).

Studies in animal models have demonstrated that pluripotent stem cells acquiring an initiating mutational event become the ‘cell of origin’ in several malignancies, including intestinal cancers 14,15). In fact, the transcriptomic and proteomic profiles of intestinal stem cells (ISC) under physiologic and APC-inactivating conditions have been successfully characterized in genetically engineered animal models that allow for lineage tracing of cells expressing Lgr5, a Wnt target gene and stem cell marker 16,17). Furthermore, inactivation of MMR function via Msh2 deletion in mouse embryonic stem cells generates a mutator phenotype causing genomic instability and accumulation of subsequent somatic mutations leading to cancer development (18), thus demonstrating characteristics of a cancer stem cell (19). Therefore, the inventors hypothesized that MMRd ISCs display a unique transcriptomic and proteomic profile that is different from their daughter cells. It is essential to understand the molecular and cellular landscape of MMRd tissue-specific stem cells in order to unravel the mechanisms behind the earliest stages of cancer initiation before macroscopic lesions are detectable, thus identifying specific targets for the development of novel cancer interception strategies and biomarkers for early detection of cancer in LS (10).

Here, the inventors present, for the first time, the whole transcriptomic and proteomic landscape of the intestinal epithelium with haploinsufficiency and complete deficiency of MMR functioning, which mimics the biology of normal colorectal and neoplastic epithelium in LS patients, respectively. This gene expression signature of MMRd ISCs derived from a genetically-engineered mouse model uncovers activated molecular pathways involved in the initiation and early steps of MMRd colorectal carcinogenesis.

### B. Materials and Methods

Mice. C57BL/6J strain of conditional knockout mice for MMR gene Msh2 (Msh2^{LoxP/LoxP}) were crossed with Villin-Cre (VC) transgene expressing mice to obtain VC-Msh2^{LoxP/LoxP} mice (20). The inventors further crossed VC-Msh2^{LoxP/LoxP} mice with a reporter Lgr5^{EGFP-IRES-creERT2} mice to track and isolate Lgr5^{EGFP+} stem cells by flow cytometry (16). The inventors generated Lgr5^{EGFP-IRES-creERT2}; VC- Msh2^{LoxP/LoxP} as Msh2 null mice in the intestine (herein referred as Msh2-KO), Lgr5^{EGFP-IRES-creERT2}; VC-Msh2^{LoxP/+} as Msh2 haplo -insufficient (Msh2-HET), and Lgr5^{EGFP-IRES-creERT2}; VC-MSh2^{+/+} as mice with wild-type Msh2 function (Msh2-WT). Also, the inventors generated intestinal organoids from Msh2-KO and Msh2-WT for validation of the expression of key genes in ISC. All animal experiments were approved by the institutional animal care and use committee (IACUC) of The University of Texas MD Anderson Cancer Center and the care of the animals was in accordance with institutional guidelines (IACUC protocol # 00000469-RN02).

Crypt isolation from small intestine and Fluorescence-activated cell sorting (FACS). Crypts from the small intestine of 8-week-old Msh2-WT, Msh2-HET, and Msh2-KO mice were harvested, and single cells from each genotype were subjected to FACS to isolate Lgr5^{EGFP+} stem cells and Lgr5^{EGFP-} daughter cells.

Transcriptome and mass spectrometry analysis of mouse specimens. RNA and total cellular extracts (TCE) were isolated from ISCs (Lgr5^{EGFP+}) and daughter cells (Lgr5^{EGFP-}) from all three mouse genotypes. RNA was extracted using TRIzol (Invitrogen) and RNA isolation kit (Ambion) and then subjected to library preparation and sequenced with an Illumina HiSeq4000 instrument. TCE were analyzed using tandem mass spectrometry. Detailed steps in the analysis of RNA-seq and proteomics along with in-depth bioinformatics analysis for differential gene expression and gene set enrichment analysis (GSEA) followed standard protocols and pipelines previously described (21).

Transcriptomic analysis of human samples and validation of MMR-deficient stem cell expression signatures. Tissue samples were acquired through endoscopic biopsies during routine screening colonoscopies from a total of 17 Familial Adenomatous Polyposis (FAP) patients (17 paired adenoma and normal mucosa) and 27 LS patients (11 matched tumor/adenomas and normal mucosa, 3 unmatched tumor/adenomas, and 18 with unmatched normal mucosa, Table S6). All patients were followed at The University of Texas MD Anderson Cancer Center (UTMDACC) for routine surveillance care. Written informed consent was obtained from all study participants, and the UTMDACC Institutional Review Board (IRB) approved this study (IRB #PA12-0327). Total RNA was isolated from tissues that have been flash-frozen or preserved in RNALater and subjected to mRNA sequencing (mRNAseq). This human mRNAseq data set from colorectal normal mucosa, polyp, and tumor samples were used for validation of the gene signatures obtained in mice. FAP normal mucosa and polyps were selected as the human counterparts of Msh2-WT mice (MMR-proficient), LS normal mucosa as Msh2-HET (MMR-haploinsufficient), while LS polyp and tumor samples that were hypermutant (mutation rate≥10/Mb estimated by whole-exome sequencing) or displayed MMRd by microsatellite instability (MSI) via PCR or immunohistochemistry of MMR protein as ‘Msh2-K0’ (MMRd). In addition, transcriptomic data from organoids of normal mucosa from LS (22) and sporadic CRC patients (23) were used to further validate the MMR-haploinsufficient expression signature.

Gene expression analysis and Chromatin Immunoprecipitation (ChIP) assays. Total RNA from ISCs and organoids isolated from the different genotypes as well as both Lgr5^{EGFP+} and Lgr5^{EGFP-} fractions were analyzed by qRT-PCR for validation of the expression of critical genes following previously described methods (22). To assess if SPP1 gene expression was epigenetically regulated by histone H3 lysine 27 methylation (H3K27me3), ChIP assays were performed in chromatin extracts of the mismatch repair proficient (MMRp) CRC cell line SW620 and the MMRd endometrial cancer cell line HEC59, which harbors bi-allelic inactivating MSH2 mutations. Primer sequences are included in Table S7.

Immunofluorescence and imaging of mouse tissues. Freshly extracted small intestine from 8-week-old mice from Msh2-WT, Msh2-HET, or Msh2-KO were used to stain Lgr5 (GFP+) cells as a marker to visualize ISC and the expression of SPP1 (Table S8).

Fluorescent multiplex immunohistochemistry (IHC) staining of human specimens and automated quantitative imaging. Formalin-Fixed Paraffin-Embedded (FFPE) tissue specimens of uninvolved normal colorectal mucosa (N=6), tubular/tubulovillius adenoma (N=6), and invasive adenocarcinoma (N=3) from a total of 8 LS patients were used (Table S9). Unstained slides were processed as described above and stained with antibodies against human LGR5 and SPP1. Manual fluorescent multiplex IHC staining was performed following a validated protocol using antibodies and reagents listed in Table S8.

Statistical analysis. Comparisons between two experimental groups were performed with GraphPad Prism using Student' s unpaired t-test, and among more than two experimental groups were analyzed using one-way ANOVA with Tukey's post-hoc analysis for multiple comparisons. The data are expressed as means ±SD from three technical replicates and three independent experiments.

C. Results

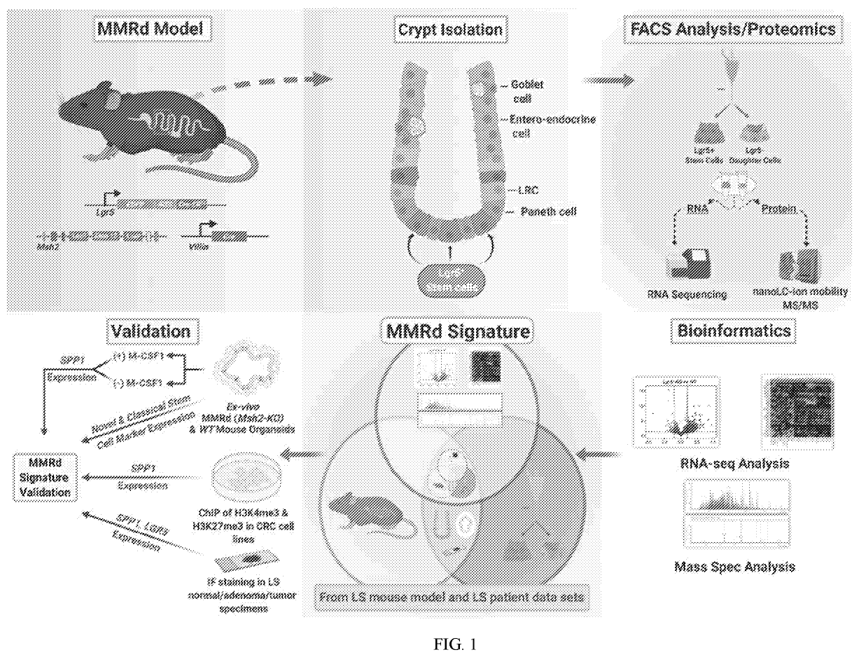

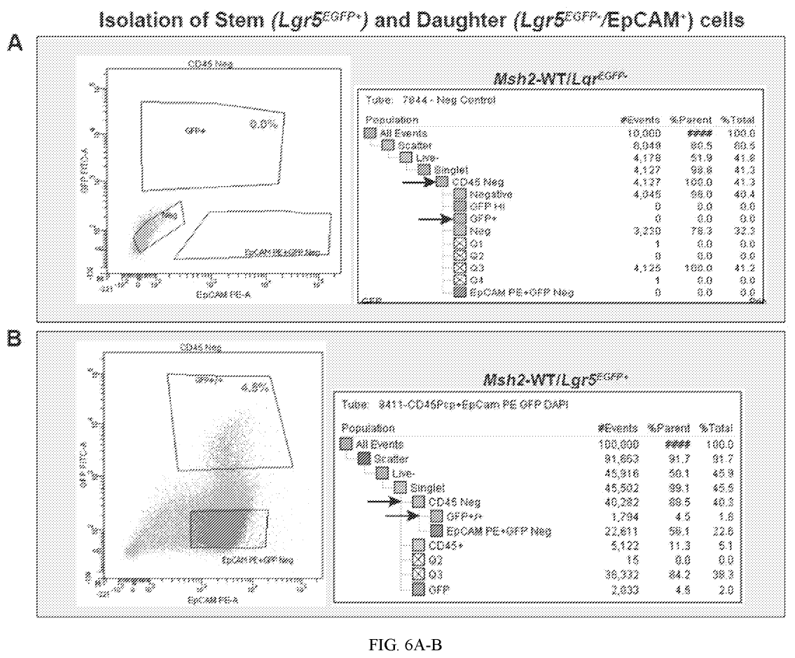

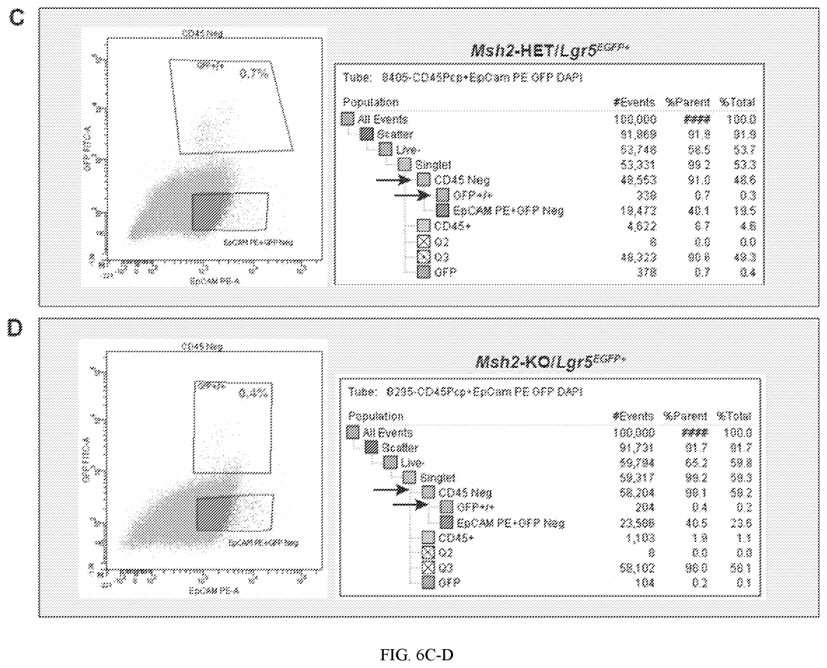

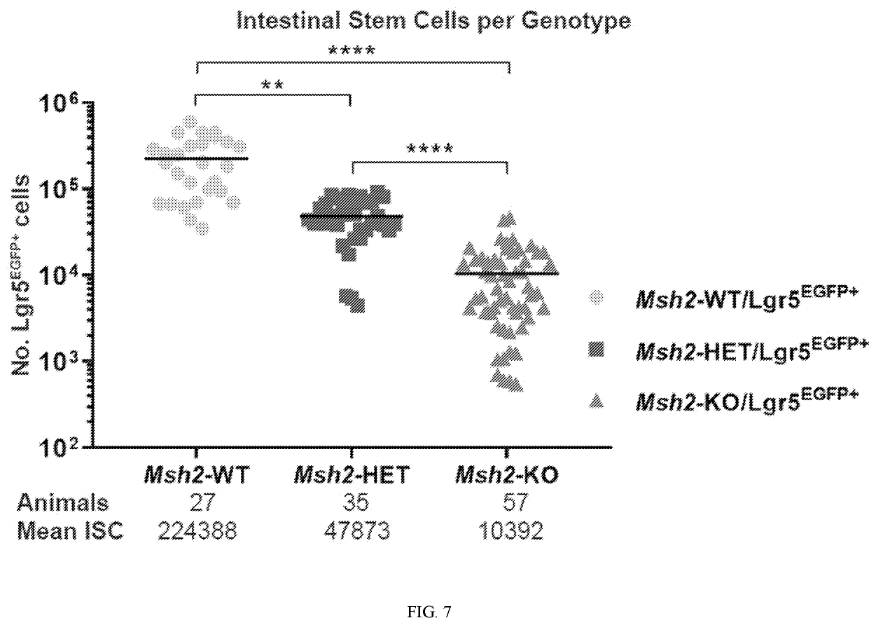

Isolation of MMRd mouse ISC. To understand the biology of MMRd ISCs at the earliest stage of carcinogenesis, the inventors crossed a mouse model of intestinal tissue-specific (Villin-Cre, VC) inactivation of the MMR function via deletion of the essential ATPase domain of Msh2 in exon 12 (Msh2^{LoxP/LoxP} , thus resulting in VC-Msh2^{LoxP/LoxP}) with another mouse line (Lgr5^{EGFP-IRES-creERT2}) that allowed the isolation and tracing of ISCs expressing the validated stem cell marker Lgr5. Then, the inventors isolated ISCs from the entire small intestine of the following mouse genotypes to model different clinical scenarios: Lgr5^{EGFP-IRES-creERT2+}; VC-Msh2^{+/+} (Msh2-WT) as a counterpart of ISC from the sporadic normal colorectal mucosa; Lgr5^{EGFP-IRES-creERT2+}; VC-Msh2^{LoxP/+} (Msh2-HET) from normal colorectal mucosa of LS patients; and Lgr5^{EGFP-IRES-creERT2+}; VC-Msh2^{LoxP/LoxP} (Msh2-KO) from early premalignant lesions of LS patients, as well as their corresponding daughter cells that were Lgr5^{EGFP-IRES-creERT2-} (FIG. 1). FACS was optimized using crypt preparations from GFP negative mice to specifically isolate epithelial cells expressing high levels of GFP (GFP^{hi}) that were considered as Lgr5^{EGFP+} ISCs and GFP⁻/EpCAM⁺ cells (Lgr5^{EGFP-}) as daughter (non-stem) cells. Cell fractions excluded lymphocytes by labeling total crypt cells with CD45 antibody (FIG. 6). A cohort of 119 mice was used to isolate a sufficient number of stem cells that afforded extraction of RNA and protein for transcriptomics and proteomics analyses, respectively. From a total of 27 Msh2-WT mice, the inventors obtained a mean of 224,388 Lgr5^{EGFP+} cells per mice; 35 Msh2-HET rendered a mean of 47,873 Lgr5^{EGFP+} cells; and 57 Msh2-KO a mean of 10,392 Lgr5^{EGFP+} cells. The inventors observed that the number of stem cells recovered for each genotype decreased exponentially with the deletion of each Msh2 allele (FIG. 7), which could possibly be due to premature differentiation of ISC upon deletion of Msh2 allele.

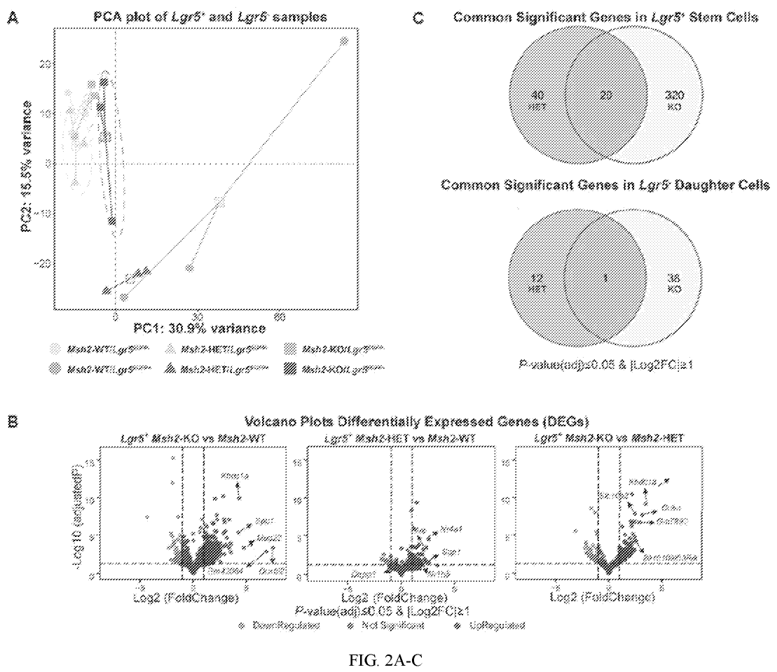

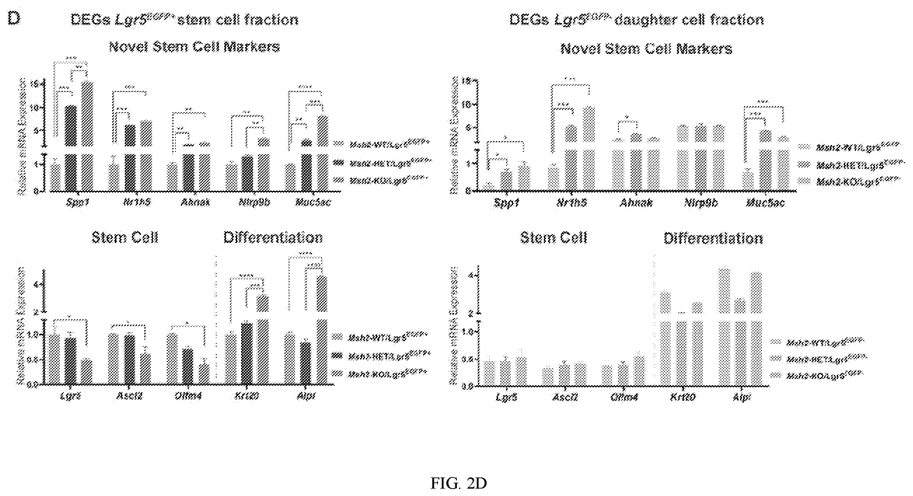

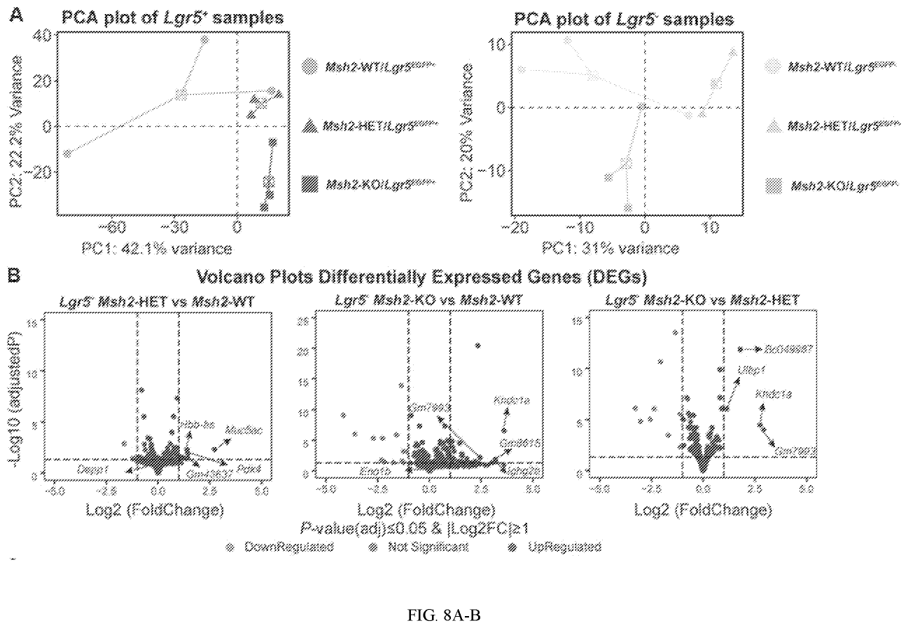

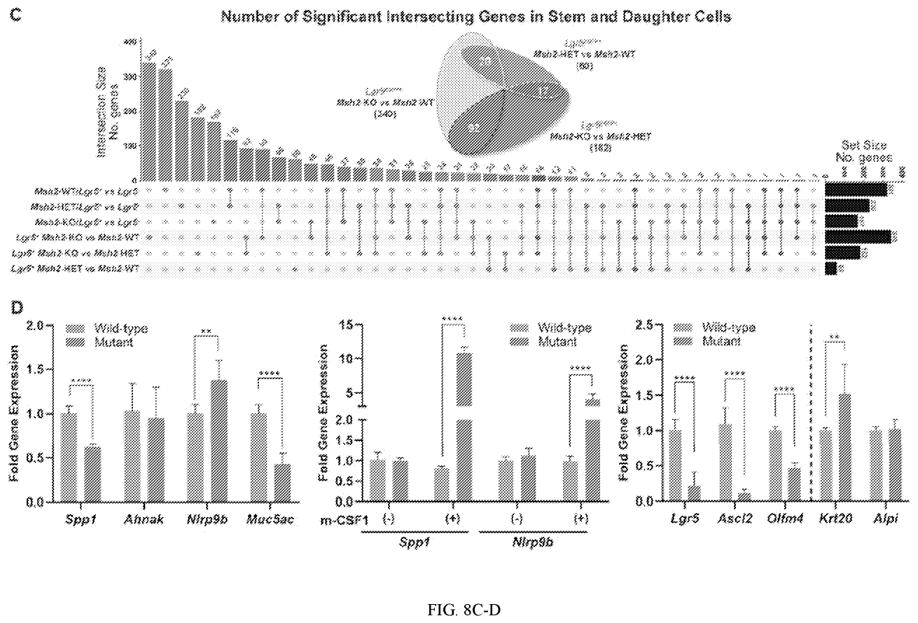

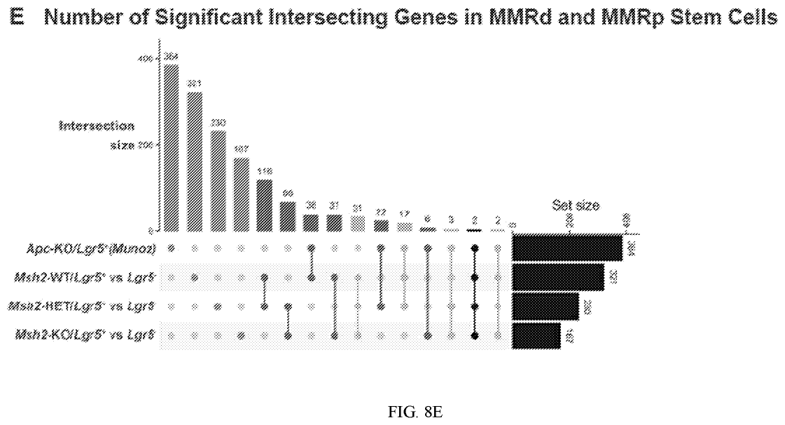

Transcriptomic profile of MMR haploinsufficient and MMRd stem and non-stem cells. The inventors identified the transcriptomes of Lgr5⁺ ISCs (Lgr5^{EGFP+}) and their daughter cells (Lgr5^{EGFP-}) in all three mouse genotypes using next-generation whole transcriptomics followed by principal component analysis (PCA). The transcriptome of Lgr5^{EGFP+} cells of Msh2-KO clustered with daughter cells (Lgr5^{EGFP-} fractions) of all three genotypes (FIG. 2A). However, each genotype showed a profile that was distinct from the others when samples were separately analyzed based on stem and daughter cell phenotypes (FIG. 8A). Then, the inventors examined the transcriptional differences of MMR haploinsufficient and MMRd ISCs (Msh2-HET and Msh2-KO mice, respectively) to MMRp ISCs (which were represented by Msh2-WT mice and were the comparator for these analyses), and their corresponding daughter cells (differentiated non-stem cells). The comparison of the transcriptomes of Lgr5^{EGFP+} Msh2-KO and Msh2-WT stem cells identified a total of 340 significantly dysregulated genes (284 upregulated and 56 downregulated, FIG. 2B), thus defining an expression profile of MMRd ISC. In contrast, the inventors observed only 39 genes significantly dysregulated in Lgr5^{EGFP-} non-stem cells (FIG. 8B). Then, a comparison of Lgr5^{EGFP+} Msh2-HET and Msh2-WT stem cells rendered a gene profile for MMR haploinsufficiency with a total of 60 genes differentially dysregulated (50 upregulated and 10 downregulated, FIG. 2B) and the same comparison of Lgr5^{EGFP-} daughter cells observed 131 genes (FIG. 8B). When the inventors changed the reference and compared the transcriptomes of Msh2-KO and Msh2-HET, the inventors observed a total of 182 genes differentially expressed (131 upregulated and 51 downregulated) in the stem cells (FIG. 2B) and 13 genes in Lgr5 EGFP non-stem cell fractions (FIG. 8C). The inventors observed that 20 genes were dysregulated in common between Lgr5^{EGFP+} stem cells of Msh2-HET and Msh2-KO (FIG. 2C and Table 1), whereas only one gene was commonly dysregulated within Lgr5 EGFP daughter cells of Msh2-HET and Msh2-KO when compared to Msh2-WT. In order to summarize the number of genes expressed in stem and daughter cells as well as the intersection among the different genotypes (Msh2 status) and cell types (Lgr5^{EGFP+} or Lgr5^{EGFP-}), the inventors generated an UpSet plot matrix that provides a visualization at a glance of these numbers (FIG. 8C). Overall, the gene profile showing the largest differences was between Msh2-KO and -WT stem cells, and the one with the closest expression was between stem and differentiated cells of the Msh2-KO genotype (FIG. 8C). These results indicate that the loss of one or both alleles of Msh2 induces a unique transcriptional profile that perturbs the biology of MMRd ISC. In addition, the transcriptome of Lgr5^{EGFP+} stem cells of Msh2-KO clustered with daughter cells (Lgr5^{EGFP-} fractions) of all three genotypes in the PCA plot and also displayed the lowest amount of differentially expressed genes compared to their daughter cells, thus indicating that a complete loss of Msh2 may lead to premature differentiation of ISC.

Validation of stem and non-stem cell specific genes in MMRd ISCs and daughter cells. The inventors assessed dysregulated genes that overlapped between Msh2-HET and Msh2-KO in Lgr5^{EGFP+} stem cells and Lgr5^{EGFP-} non-stem cells using qRT-PCR (Table 1). These markers were selected based on their potential roles in tumorigenesis and the correlation between their level of dysregulation and Msh2 allele dosage. The inventors evaluated a total of four markers of stem cells (Spp1, Nr1h5, Ahnak, and Nlrp9b) and one of daughter cells (Muc5ac). Overall, all of them were confirmed to be expressed in both Lgr5^{EGFP+} and Lgr5^{EGFP-} cells of both Msh2-HET or Msh2-KO mice. Moreover, they were significantly upregulated in stem cells and their expression correlated with the Msh2 allele dosage (FIG. 2D, upper left panel). Of note, the inventors observed a high level of upregulation of the marker Spp1 in both Msh2-HET (10-fold) and Msh2-KO (15-fold) stem cells when compared to Msh2-WT. In addition, the relative expression of Spp1 was significantly lower in Lgr5^{EGFP-} daughter cells than in Lgr5^{EGFP+} stem cells within each respective genotype. In contrast, the expression of Nr1h5 showed significant differences in daughter cells across genotypes, while Ahnak and Nlrp9b did not show differences across genotypes (FIG. 2D, upper right panel). In regards to Muc5ac, there were significant differences among daughter cells across different genotypes that were also seen among stem cells, thus making this marker relatively non-specific of cell-of-origin. The inventors attempted to validate the gene expression of the novel stem cell markers observed in the Lgr5^{EGFP+} stem cells using Msh2-KO and Msh2-WT mouse organoids as an ex vivo model of the stem cell compartment of the intestinal crypt. Initially, the inventors did not confirm the same trends observed in sorted cells (FIG. 8D, left panel). Therefore, based on the function of these genes, the inventors reasoned that their expression could be influenced by the immune environment and surrounding stem cell niche. The inventors repeated the expression assessment after stimulating the organoids for 24 hours with colony-stimulating factor 1 (M-CSF1) to recreate cues received by stem cells. Under these conditions, the inventors observed a significant upregulation of Spp1 and Nlrp9b expression in Msh2-KO organoids and no significant changes in the Msh2-WT counterparts (FIG. 8D, center panel). These results are consistent with the expression data acquired in vivo and strongly suggest that Spp1 and Nlrp9b expression levels were significantly and specifically enhanced in MMRd ISCs upon interaction with the stem cell niche, including immune cells, thus highlighting its role as a potential biomarker in MMRd carcinogenesis.

Since the transcriptomes derived from Msh2-KO Lgr5^{EGFP+} ISCs clustered together with daughter cells of all genotypes, the inventors hypothesized that Msh2-KO ISCs lost their stem-ness and underwent premature differentiation. Based on this observation, the inventors performed qRT-PCR analysis of differentiation-specific genes including Krt20 and Alpi (markers for enterocytes), and Muc2 (marker for Goblet cells) and stem cell markers, Ascl2 and Olfm4 in intestinal samples from mice of the three genotypes. The inventors confirmed a significant decrease in expression of stem cell markers, Lgr5, Ascl2, and Olfin4 in stem cells from Msh2-KO mice compared to those in Msh2-WT mice. Subsequently, the inventors observed a strong stimulation in the expression levels of enterocyte markers, Krt20 and Alpi, in the stem cells of Msh2-KO mice (FIG. 2D lower left panel). These results were confirmed ex vivo in mouse organoids that showed downregulation of surface stem cell markers and upregulation of differentiation signals (FIG. 8D, right panel). In line with these results, no significant differences were observed in the expression of these markers in Lgr5^{EGFP-} cells across all genotypes (FIG. 2D lower right panel). Therefore, these results indicate that loss of MMR function influences ISC homeostasis and promotes premature differentiation of ISCs.

Proteomic profile of MMR haploinsufficient and MMR deficient stem and non-stem cells. To assess the proteomic profile, the inventors isolated total cellular proteins from stem and non-stem fractions of Msh2-WT, Msh2-HET, and Msh2-KO mice and performed tandem mass spectrometry (MS/MS). The inventors identified an average of 1238 gene counts (proteins) from total cell extracts of stem cells from Msh2-WT (Table S1). A total of 797 and 830 proteins were observed from equal amounts of total protein of stem cells from Msh2-HET and Msh2-KO mice, respectively. The number of proteins identified from non-stem cells was higher than stem cell fractions observed in each genotype. Individual analysis of fold change expression obtained directly from mean spectral counts of Lgr5^{EGFP+} ISCs revealed high levels of differentiation markers such as Krt20 (5.5-fold) and Fabpl (5.8-fold) in Msh2-HET Lgr5^{EGFP+} cells (Table S2) and even higher in Msh2-KO Lgr5^{EGFP+} (Krt20, 6.5-fold; Fabp1, 7.3-fold; Fabp2, 2.0-fold, Table S3) compared to Lgr5^{EGFP+} Msh2-WT cells. The inventors also observed enrichment for proteins that are involved in cancer progression and migration, such as carbonic anhydrase 1 (Car1, 10.89-fold in Msh2-HET, and 21-fold in Msh2-KO) and actin-binding protein Gelsolin (Gsn, 2.5-fold in Msh2-HET and 9.8-fold in Msh2-KO) (24, 25). Thus, the proteomic expression of MMRd stem cells showed enrichment for cancer-associated markers that are involved in their malignant transformation.

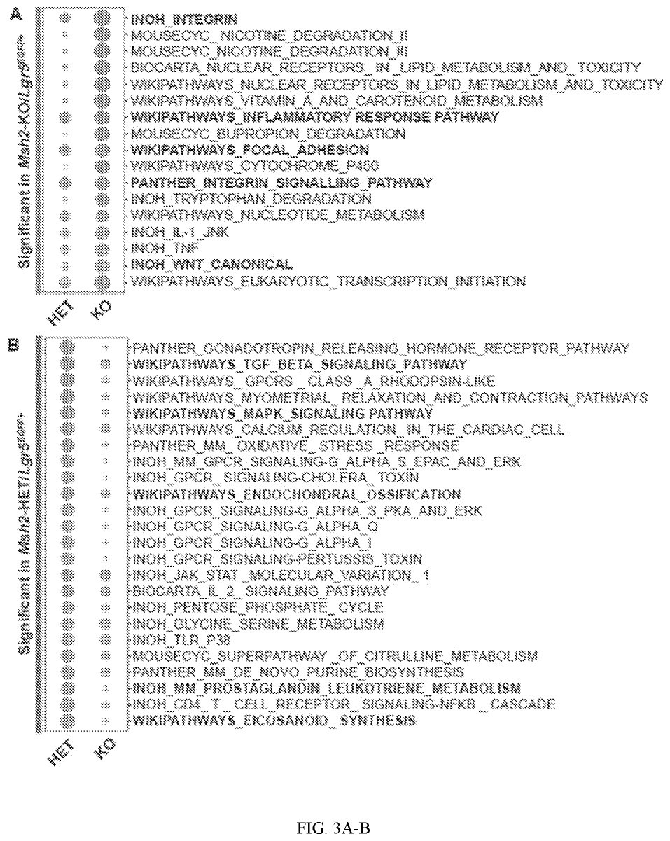

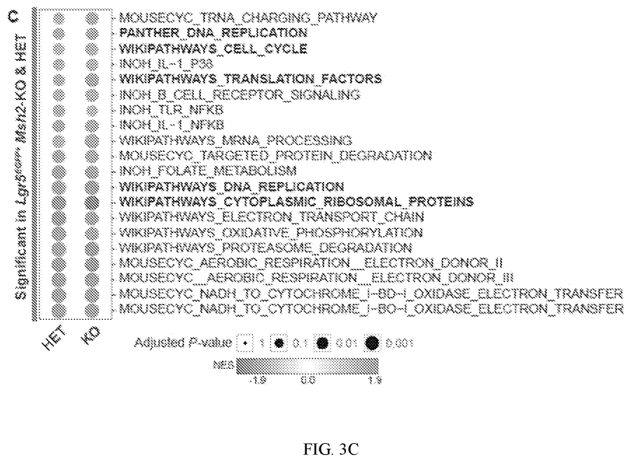

Generation of a molecular profile to define a gene signature of MMRd. Using the transcriptomic and proteomic profiles of Lgr5^{EGFP+} and Lgr5^{EGFP-} cells for each genotype, the inventors generated a molecular profile that defines a gene signature for MMR haploinsufficiency and MMRd. First, the inventors compared the expression data of Lgr5^{EGFP+} ISCs obtained from Msh2-KO to Msh2-WT mice and excluded those genes expressed commonly in Lgr5^{EGFP-} fractions in order to generate a list of unique genes that specifically represent LS colorectal neoplasia (pre-cancers and tumors), which is characterized by a complete MMRd. The inventors observed a total of 48 differentially expressed genes (Table 2). The same approach was applied to the analysis of the comparison of the transcriptome of Msh2-HET to Msh2-WT to generate a list of genes exclusively and differentially expressed in MMR haploinsufficient ISCs. This list reflected the expression patterns of LS normal colorectal mucosa and a total of 5 differentially expressed genes were detected (Table 2). GSEA highlighted several relevant pathways in MMRd ISC biology. Among Lgr5^{EGFP+} cells from Msh2-KO, the top observed pathways were related to Integrin Signaling, Focal Adhesion, and Inflammatory Response. Interestingly, the inventors also observed downregulation of the WNT pathway (FIG. 3A). The top enriched pathways in the Msh2-HET ISCs were TGFI3 signaling, mitogen-activated protein kinase (MAPK) signaling, and Endochondral Ossification (FIG. 3B). The inventors found that the pro-inflammatory gene sets, Prostaglandin-Leukotriene Metabolism and Eicosanoids Synthesis, were depleted in Msh2-HET. In addition, the inventors observed that gene sets for Cytoplasmic Ribosomal Proteins were downregulated in both Msh2-HET and Msh2-KO ISCs (FIG. 3C). Finally, the inventors compared the gene signatures reflecting different stages of MMR carcinogenesis with the previously published profile of mouse APC-driven stem cells¹⁷. The inventors observed a minimal degree of overlap with the Msh2-KO signature (only 6 genes) and increased numbers of genes shared with Msh2-HET and Msh2-WT, thus consistent with the expectation that APC-driven carcinogenesis overlaps minimally with MMRd during the earliest stages (FIG. 8E).

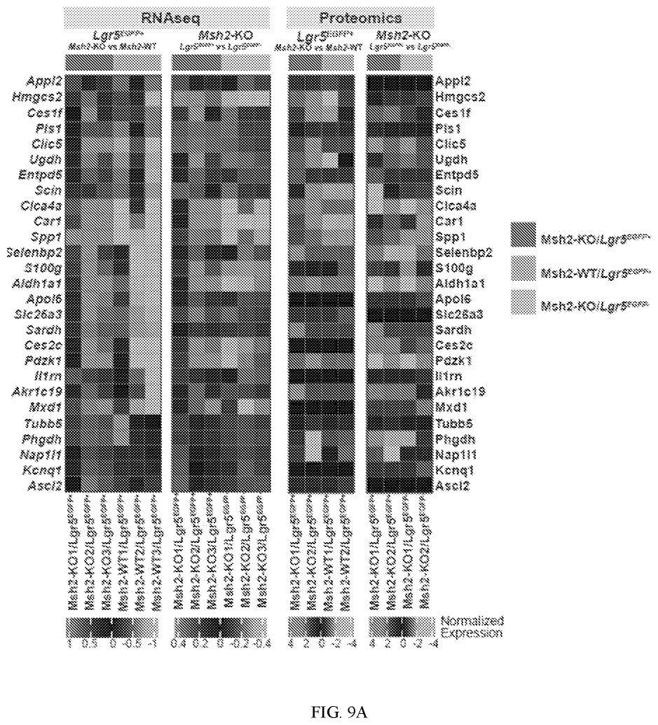

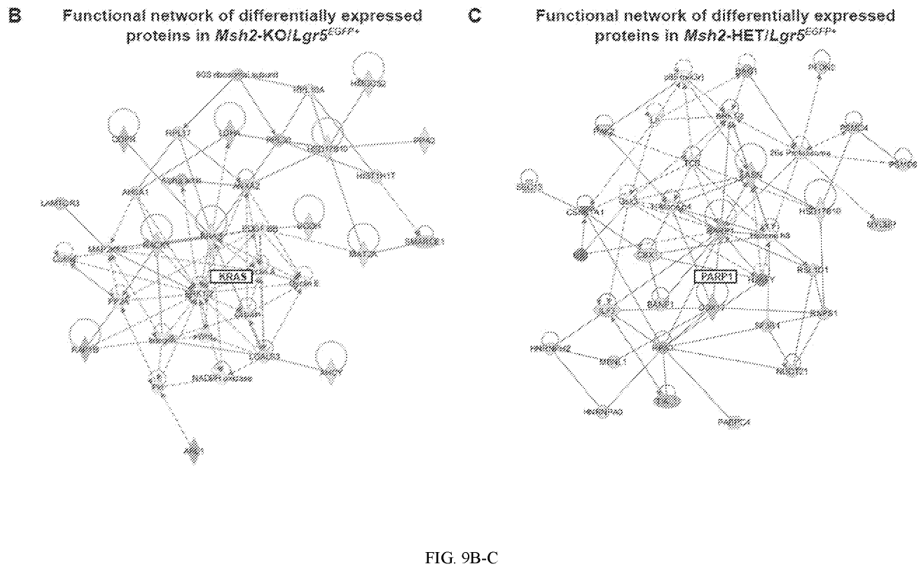

Then, the inventors generated a unique list of 78 signature proteins for Msh2-KO (MMRd) as ratio of protein expression from 130 proteins found between Lgr5^{EGFP+} and Lgr5^{EGFP-} cells of Msh2-KO mice and also from the ratio of protein expression from 117 proteins found between Lgr5^{EGFP+} cells of Msh2-KO and Msh2-WT (Table S4). From the proteomic analysis, 27 proteins were found to overlap with 197 signature genes from mRNAseq analysis for Msh2-KO (FDR≤0.05, FIG. 9A). Of note, the mass spectrometry results indicated that Spp 1 protein was found to be expressed only in Lgr5^{EGFP+} cell fractions of Msh2-KO animals. In addition, the inventors analyzed pathway enrichment using Ingenuity Pathway Analysis (IPA) observing a representative network of proteins related to cellular development, cellular growth, and proliferation as top affected pathways with expression of KRAS as a pivotal network (FIG. 9B). Similarly, a unique list of 52 signature proteins for Msh2-HET was derived from comparing the ratio of protein expression from 235 proteins found between Lgr5^{EGFP+} and Lgr5^{EGFP-} cells of Msh2-HET mice and also from the ratio of protein expression from 187 proteins found between only Lgr5^{EGFP+} cells of Msh2-HET and Msh2-WT (Table SS). DNA replication, recombination and repair, as well as RNA post-transcriptional modification pathways stood out from the Msh2-HET protein profile, which was PARP1 centered (FIG. 9C). Overall, the Msh2-HET and Msh2-KO protein signatures revealed potentially interesting candidates that merits consideration for its influence on carcinogenesis in MMR deficiency.

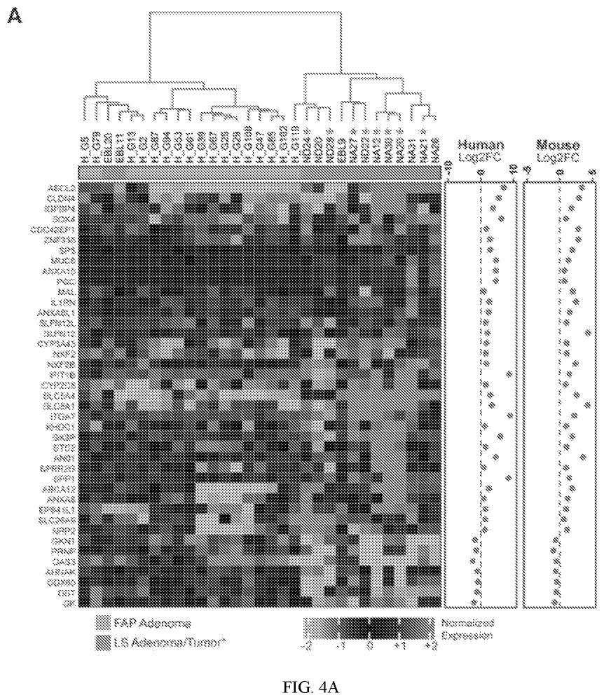

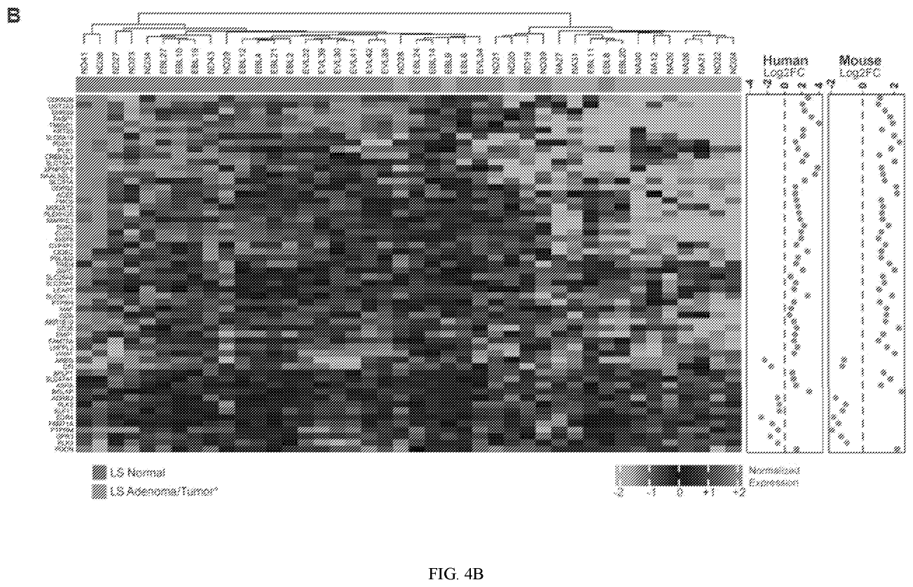

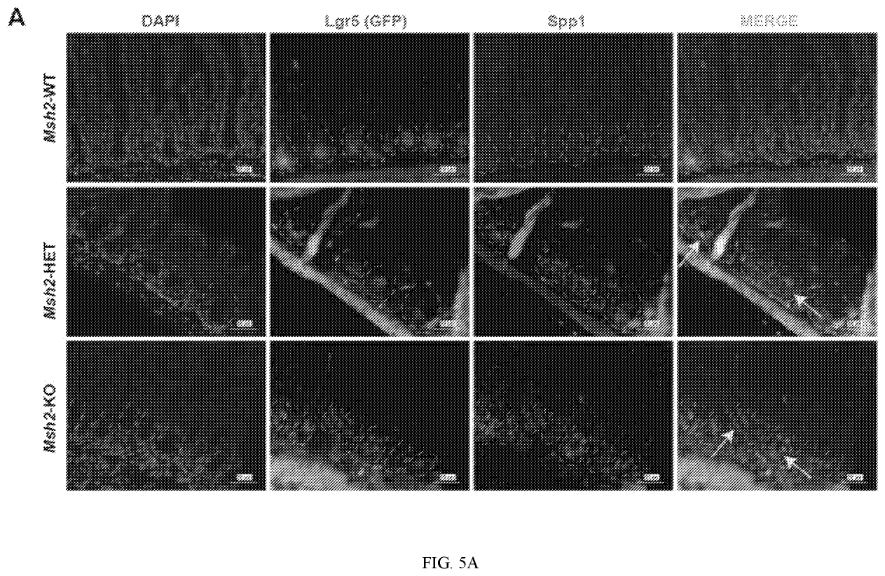

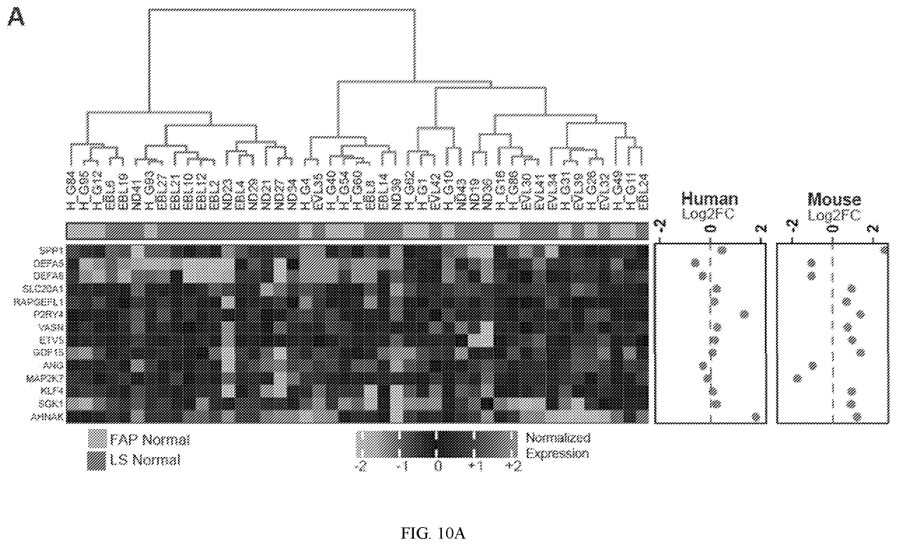

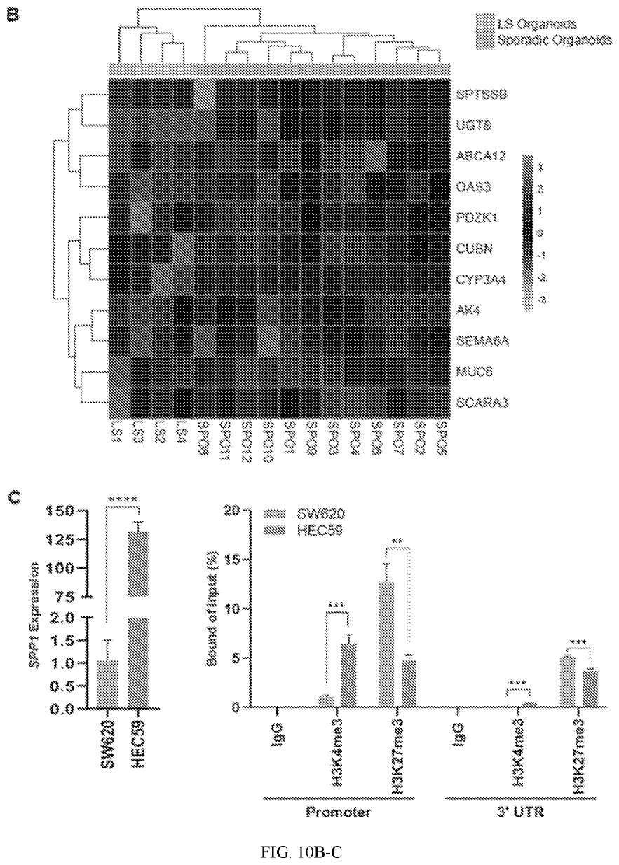

Validation of the MMRd stem cell signature in LS human specimens. To assess the biological significance of the MMRd and MMR-haploinsufficient gene signatures derived from ISC in mice, the inventors applied both signatures to whole transcriptomics of normal colorectal mucosa and neoplastic lesions (both adenomas and tumors) from a cohort of LS and FAP patients (as MMRp controls; Table S6). For the Msh2-KO signature, the inventors observed that 41 genes out of 197 signature genes were still significantly dysregulated (FDR≤0.05) and had the same fold change direction in LS hyper-mutant adenomas and tumors. This confirmed that these genes represent and recapitulate the biology of cells derived from an MMRd progenitor. Together, these 41 dysregulated genes were able to clearly separate pre-cancers and early-stage tumors in LS and FAP patients into two distinct clusters, with only two LS samples misclassified (FIG. 4A). For the MMR-haploinsufficient signature (Msh2-HET signature), the inventors had to relax the criteria because only one gene out of 27 (AHNAK) was significantly dysregulated and had the same fold change direction. Therefore, the inventors examined 14 genes that had the same fold change direction in mouse and human data sets. Unsupervised hierarchical clustering observed two groups of samples with one group integrated by mostly LS samples with the exception of 4 lesions (FIG. 10A). Of note, SPP1 showed a trend towards being upregulated in LS normal mucosa. Overall, the MMRd gene signature (Msh2-KO) was able to correctly classify neoplasms that are MMRd and therefore contain biomarker information that recapitulated early stages of MMRd carcinogenesis in humans. Then, the inventors investigated if the signature comparing Msh2-KO and Msh2-HET was able to distinguish LS pre-cancers and early-stage tumors from normal mucosa. The original signature from mouse ISC contained a total of 182 genes (FIG. 8C) and 56 genes retained statistical significance in human and shared the same fold change direction. The resulting gene profile was able to separate the samples into two groups with only three neoplastic lesions misclassified, thus showing a strong performance (FIG. 4B). Finally, the inventors combined together with the MMRd and haploinsufficient signatures (Table 2) to validate its performance to differentiate the expression patterns of human organoids derived from the normal mucosa of both LS carriers (22) and patients diagnosed with sporadic CRC (as normal mucosa controls) (23). The inventors observed that an 11-gene set of significantly expressed genes with corresponding human orthologs were able to precisely differentiate and segregate colorectal normal mucosa organoids of LS from sporadic individuals (FIG. 10B).

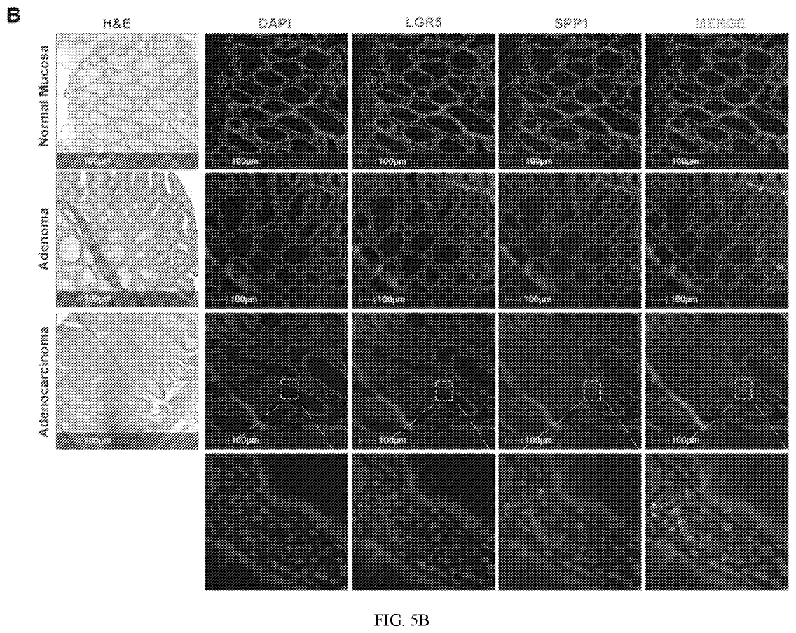

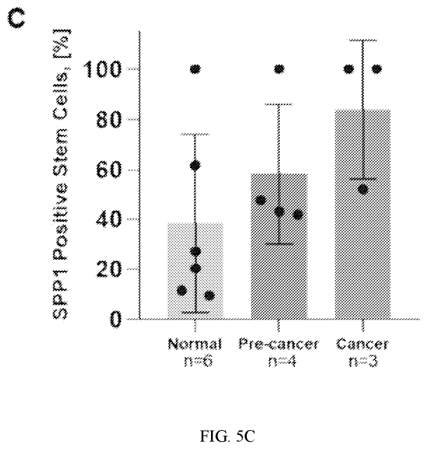

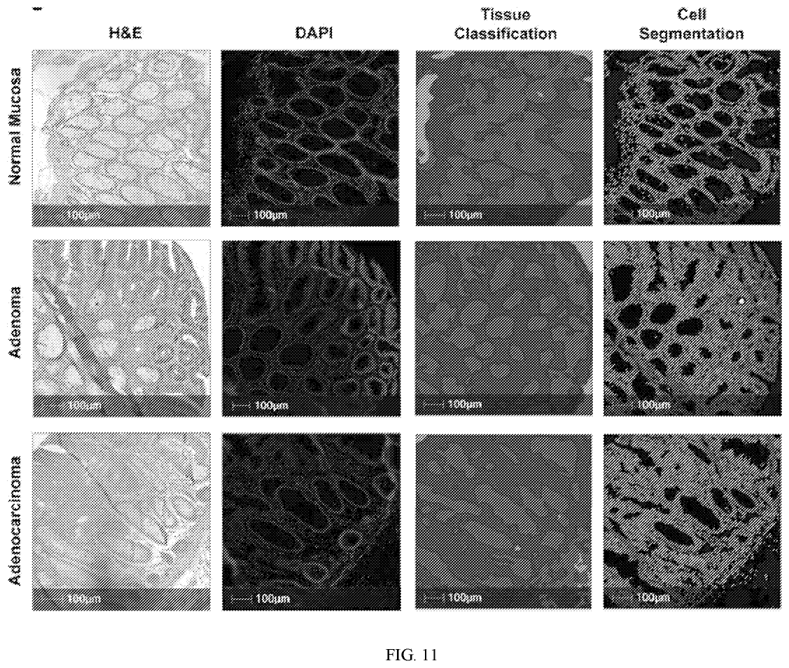

Expression of Spp1 in MMR-deficient mouse ISCs and LS patient specimens. The combined transcriptomics and proteomics analysis indicated that Spp1 expression is upregulated in ISCs of both MMRd mice and LS patients. To gain mechanistic insights in epigenetic regulation of SPP1, ChIP assays were performed in MMRp and MMRd cell line models. These results indicated that levels of H3K27me3 epigenetically regulate the expression of SPP1 as a function of the MMR status (FIG. 10C). Then, to further confirm the expression of SPP1 in stem cells (Lgr5^{EGFP+} cells), the inventors performed IHC in intestinal tissue sections of Msh2-WT, Msh2-HET, Msh2-KO mice using antibodies against Spp1 and GFP. Since GFP expression is under control of the Lgr5 promoter in mice, the level of GFP staining correlates to the level of Lgr5 cells. The inventors observed enhanced staining of Spp1 in crypts of Msh2-HET and Msh2-KO mice that co-localized with Lgr5 cells (GFP⁺) located at the base of the crypts, thus confirming the upregulation of Spp1 in ISCs of MMR haplo-insufficient and MMRd tissues (FIG. SA). Finally, the inventors examined the levels of SPP1 and LGRS in a series of LS samples (Table S9) representing sequential steps of colorectal carcinogenesis: normal mucosa (n=6), pre-cancers (n=4, tubular adenomas), and tumors (n=3, adenocarcinomas). Quantitative imaging and analysis of single- and double-positive cells for SPP1 and LGRS (FIG. 11) showed the highest proportion of co-expressing stem cells (double-positive cells) in cancers compared to adjacent normal colorectal mucosa and pre-cancers (non-statistically significant trend, P-value=0.15 cancer vs. normal, FIG. 5B and 5C). Despite the limited number of samples available for analysis, these results are consistent with the data from the LS mouse model, thus suggesting a potential role of SPP1 in the progression of colorectal MMRd carcinogenesis.

### D. Discussion

In this study, the inventors have used a tissue-specific mouse model of LS to identify, for the first time, the transcriptomic and proteomic profiles of ISCs displaying MMRd. The inventors observed a significant loss of Lgr5⁺ stem cells upon deletion of each Msh2 allele, which posed additional technical challenges as the inventors required a large number of animals to obtain sufficient numbers of cells to perform mRNAseq and mass spec analyses. The inventors validated this observation in MMRd ISCs and intestinal organoids, which revealed a loss of stemness reflected by downregulation of Lgr5, Ascl2, and Olfm4, and upregulation of the differentiation-specific markers Krt20, Alpi, and Muc2. Therefore, theseresults suggest that stem cells from Msh2-KO prematurely exhibit a differentiated phenotype, as evident by the fact that MMRd stem cells clustered together with differentiated cells of all genotypes regardless of their MMR status. It is plausible that stem cells trigger a natural epigenetic response towards differentiation to avoid malignant transformation. In fact, this mechanism has been therapeutically exploited in the treatment of leukemia, where ‘differentiation therapies’ are used to treat Acute Promyelocytic Leukemia (26). More relevant for the disease context was the observation that inhibition of R-Spondin in CRC, a ligand for Lgr5 receptor, led to in-vivo differentiation and loss of stem-cell function 27,28).

Enrichment analysis has pointed towards the dysregulation of other cellular pathways associated with the loss of stemness in MMRd ISC that could drive their transformation into a cancer stem cell phenotype (19). The inventors observed the downregulation of ribosomal proteins such as RPS7, which has been reported to act as a tumor suppressor gene inhibiting proliferation by decreasing hypoxia-inducible factor-mediated glycolysis in CRC (29), and RPS14, which has been reported to play a significant role in cell proliferation by negatively regulating the transcriptional activity of c-Myc, a key oncogene involved in colorectal carcinogenesis (30). Thus, the inventors posit that the MMR system may have a direct role on stem cell maintenance and renewal. As MMRd prompts premature differentiation of stem cells in LS, a relatively small percentage of cells persist that sustain and acquire a cancer stem cell phenotype via dysregulation of key genes from cancer promoting pathways such as those that the inventors have observed in the pathway enrichment analysis. The prematurely differentiated MMRd stem cells that have lost their stem-ness will become de-differentiated under specific conditions, which may yield pluripotency that subsequently drives the onset of carcinogenesis (31). Therefore, induction of stem cell differentiation in LS could become a potential avenue for cancer interception that warrants further investigation. Another notable finding is the downregulation of Wnt signaling in MMRd ISC. This observation confirms that the key initiating step in LS carcinogenesis is inactivation of the MMR system within aberrant crypt foci, then leading to flat pre-malignant lesions upon acquisition of additional hits in other key oncogenic drivers, with activation of Wnt signaling at later stages and only in a fraction of the pre-malignant lesions. This agrees with previous models that were based on anecdotal observations and that now can be better substantiated in these results (32).

The gene signature of MMR deficiency in stem cells is a frontier discovery that includes a unique set of genes with the potential of being a biomarker of early cancer progression. In fact, this signature is able to differentiate between MMRd and MMR-proficient neoplasia as well as organoids derived from normal tissues in the same contexts.

In conclusion, the inventors have identified a gene signature of MMRd ISCs using the transcriptomic and proteomic profiles of the stem and non-stem cells from a MMRd mouse model. The inventors have observed that the MMRd stem cell signature is able to correctly distinguish early MMRd from MMRp early neoplasia from samples of LS and FAP patients. Using systems biology approaches, molecular, and cellular studies in both mouse and human samples, the inventors identified SPP1, which qualifies as a bona fide marker of MMR deficiency in LS patients. In summary, data presented in this study advance one's understanding of ISC biology in LS patients that serves as the starting point to develop novel markers of early detection of progression of LS carcinogenesis and potential targets for cancer interception strategies in this patient population.

### E. Tables

TABLE 1
Common significant genes in stem (upper section) and daughter
cells (lower section) isolated from Msh2-HET and Msh2-KO
mice. Genes selected had FDR ≤ 0.05 and |Log2FC| ≥ 1.

| | Fold Change Lgr5^{EGFP+} | Fold Change Lgr5^{EGFP+} |
|---|---|---|
| Gene Symbol | Het vs WT | KO vs WT |
| Spp1 | 6.019 | 19.335 |
| Nr1h5 | 4.862 | 8.549 |
| Rian | 3.727 | 4.717 |
| Jchain | 3.682 | 3.763 |
| Meg3 | 3.520 | 4.305 |
| Gm28230 | 3.515 | 5.396 |
| Dgkh | 3.045 | 3.907 |
| Arhgap45 | 2.943 | 3.587 |
| Trpv3 | 2.938 | 2.771 |
| P2ry4 | 2.612 | 3.041 |
| Ccn3 | 2.564 | 2.613 |
| B230206H07Rik | 2.518 | 4.196 |
| Tchh | 2.437 | 3.435 |
| Lifr | 2.350 | 2.069 |
| Ahnak | 2.290 | 3.747 |
| Sardh | 2.236 | 3.526 |
| Cacna1h | 2.204 | 2.373 |
| Sned1 | 2.195 | 2.307 |
| Nlrp9b | 2.040 | 4.672 |
| Gm23547 | 0.361 | 0.361 |

| | Fold Change Lgr5^{EGFP-} | Fold Change Lgr5^{EGFP-} |
|---|---|---|
| | Het vs WT | KO vs WT |
| Muc5ac | 6.638 | 3.905 |

TABLE 2
List of genes defining a signature of MMR haploinsufficiency
(Msh2-HET, upper section) and MMRd (Msh2-KO, lower section).
Genes selected had FDR ≤ 0.05 and |Log2FC| ≥ 1.

| | Fold Change Lgr5^{EGFP+} | Fold Change Msh2-Het |
|---|---|---|
| Gene Symbol | Msh2-Het vs Msh2-WT | Lgr5^{EGFP+} vs Lgr5^{EGFP-} |
| Spp1 | 6.019 | 8.984 |
| Meg3 | 3.520 | 0.413 |
| P2ry4 | 2.612 | 0.442 |
| Defa5 | 0.486 | 0.318 |
| Gm49320 | 0.297 | 0.410 |

| | Fold Change Lgr5^{EGFP+} | Fold Change Msh2-KO |
|---|---|---|
| | Msh2-KO vs Msh2-WT | Lgr5^{EGFP+} vs Lgr5^{EGFP-} |
| Mup22 | 27.554 | 12.855 |
| Spp1 | 19.335 | 19.562 |
| Slc26a9 | 11.831 | 6.639 |
| Muc6 | 10.515 | 11.907 |
| Ugt8a | 10.462 | 2.432 |
| Cubn | 8.143 | 2.473 |
| Pgc | 7.723 | 32.672 |
| Aqp5 | 7.414 | 14.058 |
| Cyp2c55 | 5.917 | 3.439 |
| Abca12 | 5.825 | 3.805 |
| Slc5a4a | 5.580 | 3.023 |
| Gm28230 | 5.396 | 2.352 |
| Mal | 5.256 | 3.366 |
| Car1 | 4.989 | 2.706 |
| G6pc | 4.820 | 2.433 |
| Sprr1a | 4.709 | 2.328 |
| Slc5a12 | 4.637 | 2.482 |
| Scara3 | 4.513 | 4.933 |
| Gif | 4.484 | 13.338 |
| Slc30a10 | 4.259 | 2.472 |
| Lct | 4.215 | 2.923 |
| Sptssb | 4.087 | 2.970 |
| Sgk2 | 4.058 | 2.795 |
| Clca4a | 4.052 | 2.490 |
| Cyp3a25 | 3.804 | 2.669 |
| Gdpd2 | 3.690 | 2.173 |
| Gkn1 | 3.677 | 3.141 |
| Tmigd1 | 3.602 | 2.732 |
| Slc2a2 | 3.575 | 3.053 |
| Aldh1a1 | 3.476 | 2.309 |
| Fa2h | 3.466 | 2.378 |
| Bst1 | 3.453 | 2.303 |
| 1810065E05Rik | 3.436 | 2.567 |
| Anxa10 | 3.435 | 2.881 |
| Slc10a2 | 3.298 | 2.114 |
| Clu | 3.193 | 4.456 |
| 2010109I03Rik | 3.181 | 2.060 |
| Pdzk1 | 3.000 | 3.215 |
| Cyp2b10 | 2.804 | 3.551 |
| Mafb | 2.766 | 2.497 |
| Slc5a4b | 2.594 | 2.618 |
| Slc16a9 | 2.581 | 2.080 |
| Ifit1 | 2.473 | 2.235 |
| Oas3 | 2.346 | 2.001 |
| Ak4 | 2.264 | 2.020 |
| Sema6a | 2.225 | 2.220 |
| Slc28a1 | 2.168 | 2.053 |
| Itga7 | 2.060 | 0.000 |

TABLE S1
Mean gene and MS2 spectral counts
for each genotype in MS experiment.

| | | Gene Counts | MS2 total |
|---|---|---|---|
| No. | Genotype | (mean) | (mean) |
| 1 | Lgr5^{EGFP+} Msh2-WT | 1238 | 23682 |
| 2 | Lgr5^{EGFP-} Msh2-WT | 1330 | 33661 |
| 3 | Lgr5^{EGFP+} Msh2-HET | 797 | 14633 |
| 4 | Lgr5^{EGFP-} Msh2-HET | 1487 | 38608 |
| 5 | Lgr5^{EGFP+} Msh2-KO | 830 | 14229 |
| 6 | Lgr5^{EGFP-} Msh2-KO | 1477 | 34176 |

TABLE S2
Protein expression ratio in ISCs (Lgr5EGFP+) from Msh2-HET vs Msh2-WT and
also ISCs (Lgr5EGFP+) vs daughter cells (Lgr5EGFP−) from Msh2-HET with
their overall abundance in respective fractions as mean spectral counts (MS2).

| | Msh2-HET | Msh2-HET | | | |
|---|---|---|---|---|---|
| | Lgr5^{EGFP+} vs | Lgr5^{EGFP+} vs | Msh2-HET | Msh2-HET | Msh2-WT |
| | Msh2-WT | Lgr5^{EGFP-} | Lgr5^{EGFP+} | Lgr5^{EGFP-} | Lgr5^{EGFP+} |
| Gene | Lgr5^{EGFP+} ratio | ratio | MS2 ave | MS2 ave | MS2 ave |
| Ca1 | 11.225 | 4.933 | 182.684 | 37.030 | 16.275 |
| Car1 | 10.893 | 2.366 | 33.621 | 14.208 | 3.086 |
| Acta1 | 7.749 | 1.030 | 212.955 | 206.800 | 27.481 |
| Lgals4 | 7.215 | 1.444 | 178.158 | 123.416 | 24.692 |
| Fabp1 | 5.810 | 1.178 | 66.831 | 56.744 | 11.503 |
| Krt20 | 5.487 | 1.451 | 161.700 | 111.421 | 29.470 |
| Ces1f | 5.417 | 2.470 | 34.973 | 14.160 | 6.456 |
| Cndp2 | 3.958 | 1.487 | 107.741 | 72.477 | 27.219 |
| Lgals6 | 3.392 | 1.194 | 30.447 | 25.493 | 8.976 |
| Ndufb5 | 3.390 | 1.596 | 10.463 | 6.555 | 3.086 |
| Gm5160 | 3.191 | 3.548 | 107.447 | 30.281 | 33.668 |
| Hist1h1b | 2.941 | 8.892 | 68.477 | 7.701 | 23.283 |
| Nudcd2 | 2.934 | 5.097 | 6.583 | 1.292 | 2.244 |
| Calml4 | 2.870 | 1.300 | 31.388 | 24.153 | 10.937 |
| Magohb | 2.713 | 4.864 | 28.155 | 5.788 | 10.378 |
| Lgals3 | 2.699 | 1.795 | 105.273 | 58.657 | 38.999 |
| Acp1 | 2.663 | 4.128 | 10.463 | 2.535 | 3.929 |
| Hist1h1c | 2.571 | 9.175 | 131.253 | 14.305 | 51.047 |
| Anxa2 | 2.552 | 1.700 | 151.825 | 89.309 | 59.492 |
| Gsn | 2.544 | 1.555 | 20.690 | 13.304 | 8.134 |
| Adk | 2.514 | 1.283 | 27.508 | 21.433 | 10.944 |
| Hist1h1e | 2.498 | 13.276 | 122.613 | 9.235 | 49.086 |
| H2-Aa | 2.484 | 1.164 | 96.867 | 83.247 | 38.991 |
| Cyb5a | 2.478 | 1.580 | 93.164 | 58.979 | 37.597 |
| Cox6a1 | 2.462 | 0.933 | 35.914 | 38.507 | 14.590 |
| Hist1h4a | 2.452 | 3.686 | 55.017 | 14.927 | 22.441 |
| Pcbd1 | 2.429 | 0.885 | 13.637 | 15.403 | 5.614 |
| Prdx5 | 2.404 | 1.501 | 269.089 | 179.225 | 111.948 |
| Ppia | 2.367 | 2.489 | 224.475 | 90.173 | 94.838 |
| Alyref2 | 2.359 | 2.630 | 30.447 | 11.576 | 12.905 |
| Mtatp8 | 2.334 | 2.246 | 27.508 | 12.246 | 11.787 |
| Ccdc47 | 2.322 | 0.436 | 14.988 | 34.350 | 6.456 |
| Ahcy | 2.293 | 1.554 | 83.642 | 53.821 | 36.478 |
| ALB | 2.264 | 5.033 | 632.515 | 125.669 | 279.375 |
| Mic13 | 2.226 | 1.667 | 14.988 | 8.993 | 6.732 |
| Cox5b | 2.207 | 2.898 | 26.627 | 9.187 | 12.063 |
| Cox4i1 | 2.177 | 1.533 | 76.353 | 49.801 | 35.077 |
| Qdpr | 2.170 | 1.117 | 15.224 | 13.635 | 7.015 |
| Aldoart2 | 2.158 | 3.161 | 24.805 | 7.847 | 11.496 |
| Hagh | 2.154 | 0.762 | 19.338 | 25.365 | 8.976 |
| Slc9a3r1 | 2.140 | 1.512 | 70.240 | 46.451 | 32.826 |
| Cox5a | 2.136 | 0.816 | 39.558 | 48.461 | 18.519 |
| Eno1 | 2.123 | 1.203 | 476.107 | 395.816 | 224.264 |
| Eif3i | 2.119 | 2.578 | 46.376 | 17.986 | 21.881 |
| Cox7a2 | 2.119 | 0.778 | 26.157 | 33.631 | 12.346 |
| Uqcrfs1 | 2.112 | 0.704 | 13.637 | 19.375 | 6.456 |
| Etfb | 2.094 | 2.082 | 166.814 | 80.122 | 79.675 |
| Banf1 | 2.075 | 3.906 | 30.271 | 7.750 | 14.590 |
| Tsta3 | 2.027 | 1.310 | 22.747 | 17.364 | 11.220 |
| S100a11 | 2.010 | 1.480 | 15.224 | 10.285 | 7.575 |
| Rpl38 | 0.493 | 1.131 | 10.227 | 9.042 | 20.763 |
| Tmod3 | 0.474 | 0.713 | 15.694 | 22.006 | 33.109 |
| Srsf5 | 0.468 | 0.451 | 8.405 | 18.656 | 17.953 |
| Pfdn5 | 0.455 | 0.888 | 16.575 | 18.656 | 36.464 |
| Uqcrc1 | 0.449 | 0.491 | 34.973 | 71.234 | 77.976 |
| Ptbp1 | 0.444 | 0.496 | 80.409 | 162.099 | 180.941 |
| Atp2a2 | 0.425 | 0.595 | 29.095 | 48.937 | 68.476 |
| Erp29 | 0.424 | 0.427 | 10.227 | 23.968 | 24.118 |
| Tmpo | 0.414 | 0.563 | 85.640 | 152.203 | 207.069 |
| Cct5 | 0.413 | 0.696 | 27.920 | 40.138 | 67.598 |
| Rpl15 | 0.408 | 0.586 | 13.401 | 22.862 | 32.826 |
| Anp32a | 0.403 | 1.843 | 21.160 | 11.479 | 52.449 |
| Rpl13a-ps1 | 0.386 | 0.709 | 6.818 | 9.615 | 17.676 |
| Tubb3 | 0.360 | 0.540 | 26.803 | 49.607 | 74.379 |
| Idh2 | 0.358 | 0.571 | 19.985 | 35.020 | 55.825 |
| H2afz | 0.321 | 0.439 | 17.927 | 40.848 | 55.825 |
| Pabpc4 | 0.304 | 0.496 | 25.686 | 51.763 | 84.446 |
| Rnpep | 0.291 | 0.383 | 8.640 | 22.580 | 29.732 |
| Pgm2 | 0.277 | 0.465 | 8.170 | 17.558 | 29.456 |
| Dynlrb1 | 0.263 | 0.542 | 8.640 | 15.927 | 32.833 |
| Srrt | 0.253 | 0.277 | 14.988 | 54.143 | 59.181 |
| Phb2 | 0.233 | 0.241 | 5.231 | 21.676 | 22.441 |
| Rps18 | 0.227 | 0.771 | 8.405 | 10.906 | 37.031 |

TABLE S3
Protein expression ratio in ISCs (Lgr5EGFP+) from Msh2-KO vs Msh2-WT and
also ISCs (Lgr5EGFP+) vs daughter cells (Lgr5EGFP−) from Msh2-KO with
their overall abundance in respective fractions as mean spectral counts (MS2).

| | Msh2-KO | Msh2-KO | | | |
|---|---|---|---|---|---|
| | Lgr5^{EGFP+} vs. | Lgr5^{EGFP+} vs | Msh2-KO | Msh2-KO | Msh2-WT |
| | Msh2-WT | Lgr5^{EGFP-} | Lgr5^{EGFP+} | Lgr5^{EGFP-} | Lgr5^{EGFP+} |
| Gene | Lgr5^{EGFP+} ratio | ratio | MS2 ave | MS2 ave | MS2 ave |
| Car1 | 21.391 | 4.853 | 66.022 | 13.605 | 3.086 |
| Ca1 | 14.731 | 6.294 | 239.736 | 38.091 | 16.275 |
| Gsn | 9.823 | 10.391 | 79.896 | 7.689 | 8.134 |
| Fabp1 | 7.261 | 1.474 | 83.529 | 56.661 | 11.503 |
| Adam10 | 6.992 | 1.980 | 15.691 | 7.925 | 2.244 |
| Krt20 | 6.492 | 1.825 | 191.316 | 104.802 | 29.470 |
| Ces1f | 6.151 | 3.357 | 39.714 | 11.829 | 6.456 |
| Qdpr | 5.980 | 2.303 | 41.952 | 18.218 | 7.015 |
| Lgals4 | 4.989 | 0.774 | 123.196 | 159.100 | 24.692 |
| Acta1 | 4.832 | 0.801 | 132.781 | 165.845 | 27.481 |
| Lgals6 | 4.642 | 1.295 | 41.671 | 32.176 | 8.976 |
| Adh1 | 4.313 | 2.423 | 98.014 | 40.457 | 22.724 |
| Cndp2 | 4.141 | 1.761 | 112.720 | 63.995 | 27.219 |
| Sdha | 4.124 | 1.180 | 68.261 | 57.845 | 16.551 |
| Ndufb5 | 3.952 | 3.556 | 12.199 | 3.431 | 3.086 |
| Mydgf | 3.732 | 2.498 | 36.644 | 14.668 | 9.819 |
| Acadl | 3.726 | 1.517 | 47.026 | 30.993 | 12.622 |
| Anxa2 | 3.677 | 1.993 | 218.737 | 109.775 | 59.492 |
| Ugp2 | 3.675 | 2.905 | 75.245 | 25.905 | 20.473 |
| Wdr1 | 3.362 | 2.242 | 108.444 | 48.380 | 32.259 |
| Casp3 | 3.287 | 3.558 | 43.346 | 12.184 | 13.188 |
| Tsta3 | 3.274 | 4.501 | 36.738 | 8.162 | 11.220 |
| Hist1h1t | 3.265 | 6.725 | 90.701 | 13.488 | 27.778 |
| Ugt1a9 | 3.221 | 1.485 | 43.393 | 29.217 | 13.471 |
| S100a11 | 3.215 | 2.122 | 24.351 | 11.475 | 7.575 |
| Gm5160 | 3.211 | 4.639 | 108.115 | 23.304 | 33.668 |
| Ugt1a7c | 3.181 | 1.493 | 67.838 | 45.425 | 21.329 |
| Nudcd2 | 3.112 | 2.811 | 6.984 | 2.484 | 2.244 |
| Ldha | 3.106 | 2.208 | 490.668 | 222.271 | 157.997 |
| Gm7293 | 3.027 | 2.224 | 253.109 | 113.796 | 83.611 |
| Ahcy | 2.920 | 2.333 | 106.533 | 45.659 | 36.478 |
| Lgals3 | 2.806 | 2.628 | 109.415 | 41.639 | 38.999 |
| Pcbd1 | 2.795 | 1.458 | 15.691 | 10.765 | 5.614 |
| Cisd1 | 2.770 | 1.165 | 27.984 | 24.012 | 10.102 |
| Serpinb1a | 2.769 | 1.308 | 215.198 | 164.545 | 77.721 |
| Apoa1 | 2.696 | 3.140 | 62.764 | 19.990 | 23.283 |
| ALB | 2.663 | 5.616 | 744.044 | 132.490 | 279.375 |
| Hadh | 2.611 | 1.457 | 73.241 | 50.273 | 28.054 |
| Hist1h4a | 2.567 | 4.197 | 57.597 | 13.722 | 22.441 |
| Atp1b1 | 2.518 | 1.097 | 52.288 | 47.672 | 20.763 |
| Cdh17 | 2.500 | 1.961 | 123.431 | 62.929 | 49.376 |
| Cyb5a | 2.499 | 1.839 | 93.959 | 51.101 | 37.597 |
| Acsl5 | 2.398 | 2.109 | 97.545 | 46.251 | 40.669 |
| Ndufa4 | 2.361 | 2.780 | 47.026 | 16.914 | 19.920 |
| Idh3b | 2.360 | 1.721 | 62.905 | 36.551 | 26.653 |
| Gapdh | 2.299 | 1.993 | 416.723 | 209.124 | 181.238 |
| Suclg2 | 2.254 | 0.799 | 71.471 | 89.426 | 31.714 |
| Atp1a1 | 2.251 | 0.990 | 146.529 | 147.982 | 65.099 |
| Mlec | 2.224 | 2.454 | 41.812 | 17.036 | 18.802 |
| Vil1 | 2.222 | 1.233 | 247.504 | 200.741 | 111.403 |
| Aco2 | 2.194 | 1.885 | 174.184 | 92.389 | 79.392 |
| Vdac2 | 2.180 | 2.803 | 157.146 | 56.069 | 72.093 |
| HBA | 2.165 | 17.326 | 518.558 | 29.930 | 239.527 |
| Idi1 | 2.154 | 1.681 | 43.534 | 25.905 | 20.211 |
| Erp29 | 2.098 | 1.868 | 50.612 | 27.088 | 24.118 |
| Fabp2 | 2.036 | 0.839 | 53.682 | 63.995 | 26.362 |
| Myl6 | 2.036 | 1.308 | 74.870 | 57.252 | 36.769 |
| Pls1 | 2.023 | 1.016 | 132.231 | 130.117 | 65.375 |
| Ybx1 | 0.491 | 0.954 | 45.492 | 47.672 | 92.573 |
| Hdgf | 0.487 | 0.408 | 26.121 | 63.999 | 53.588 |
| Pabpc4 | 0.478 | 0.753 | 40.371 | 53.583 | 84.446 |
| Anxa11 | 0.469 | 1.626 | 17.508 | 10.765 | 37.307 |
| Elavl1 | 0.469 | 2.007 | 31.570 | 15.732 | 67.322 |
| Tufm | 0.459 | 0.911 | 90.983 | 99.846 | 198.376 |
| Rps25 | 0.453 | 0.941 | 27.937 | 29.692 | 61.708 |
| Tubb3 | 0.448 | 0.523 | 33.340 | 63.759 | 74.379 |
| Sptan1 | 0.447 | 0.429 | 619.327 | 1444.584 | 1385.463 |
| Hnrnpa1 | 0.445 | 0.496 | 57.550 | 115.933 | 129.306 |
| Rpl5 | 0.444 | 0.388 | 33.293 | 85.882 | 74.911 |
| Srsf7 | 0.437 | 0.542 | 6.984 | 12.894 | 15.992 |
| Nsun2 | 0.433 | 0.977 | 40.230 | 41.163 | 92.870 |
| Nit2 | 0.426 | 0.815 | 6.937 | 8.517 | 16.275 |
| Eef1d | 0.387 | 0.267 | 36.409 | 136.510 | 94.024 |
| Hnrnpd | 0.374 | 0.463 | 38.601 | 83.394 | 103.248 |
| Vwa5a | 0.369 | 0.457 | 7.031 | 15.377 | 19.078 |
| Tcp1 | 0.363 | 0.380 | 29.707 | 78.074 | 81.905 |
| Pdlim1 | 0.357 | 0.286 | 31.523 | 110.365 | 88.382 |
| Hnrnpc | 0.348 | 0.427 | 44.004 | 103.150 | 126.283 |
| Hspa1a | 0.336 | 0.525 | 26.121 | 49.801 | 77.714 |
| Tmod3 | 0.315 | 0.513 | 10.429 | 20.345 | 33.109 |
| Luc7l2 | 0.300 | 0.547 | 12.293 | 22.475 | 40.952 |
| Anp32a | 0.300 | 1.603 | 15.738 | 9.819 | 52.449 |
| Rnpep | 0.296 | 0.271 | 8.801 | 32.533 | 29.732 |
| Dynlrb1 | 0.267 | 0.851 | 8.754 | 10.291 | 32.833 |
| Stmn1 | 0.247 | 0.418 | 8.801 | 21.056 | 35.629 |
| Btf3 | 0.247 | 0.199 | 5.262 | 26.497 | 21.322 |
| Calu | 0.205 | 0.111 | 15.738 | 142.307 | 76.907 |
| Cacybp | 0.204 | 0.318 | 5.262 | 16.561 | 25.810 |
| Atp5d | 0.103 | 0.116 | 3.492 | 30.045 | 33.951 |

TABLE S4
Unique proteins in Msh2-KO gene (proteomic) signature.

| | Msh2-KO Lgr5^{EGFP+} | Msh2-KO Lgr5^{EGFP+} vs |
|---|---|---|
| Gene | vs Lgr5^{EGFP-} ratio | Msh2-WT Lgr5^{EGFP+} ratio |
| Acsl5 | 2.086 | 2.556 |
| Actb | 2.377 | 2.265 |
| Actc1 | 4.786 | 3.360 |
| Adh1 | 2.388 | 3.032 |
| Ahcy | 2.305 | 1.973 |
| Ahsa1 | 3.294 | 1.627 |
| Akr1c12 | 4.449 | 2.595 |
| Anxa2 | 1.984 | 2.356 |
| Apeh | 3.047 | 4.500 |
| Arl1 | 2.704 | 4.687 |
| Atp2a2 | 2.804 | 2.258 |
| Bzw1 | 3.103 | 3.404 |
| Ca1 | 6.158 | 2.332 |
| Car1 | 4.589 | 3.125 |
| Casp3 | 3.364 | 2.123 |
| Cct6a | 3.296 | 4.167 |
| Ces1f | 3.174 | 1.654 |
| Clic1 | 2.972 | 1.686 |
| Ddx39b | 2.023 | 1.682 |
| Ddx5 | 2.095 | 4.895 |
| Eif5a | 3.814 | 3.874 |
| Fabp6 | 2.093 | 7.983 |
| Fam98b | 3.330 | 1.876 |
| Gapdh | 1.988 | 2.030 |
| Gm5039 | 2.467 | 1.500 |
| Gm7293 | 2.214 | 2.046 |
| Gnb2 | 2.973 | 1.644 |
| Gsn | 9.310 | 5.563 |
| Hist1h1t | 6.330 | 1.890 |
| Hist1h4a | 3.980 | 1.574 |
| Hmgcll1 | 3.328 | 2.140 |
| Hmgcs2 | 2.368 | 2.212 |
| Hsd17b10 | 2.158 | 1.975 |
| Hsd17b11 | 2.423 | 7.192 |
| Kras | 4.974 | 1.591 |
| Krt71 | 2.365 | 2.060 |
| Krt84 | 2.370 | 1.527 |
| Lamtor3 | 5.323 | 2.783 |
| Ldha | 2.202 | 3.131 |
| Lgals3 | 2.590 | 1.831 |
| Mat2a | 4.453 | 2.129 |
| Mettl7a1 | 7.626 | 1.582 |
| Mfsd2b | 3.637 | 1.902 |
| Mp68 | 2.923 | 1.905 |
| Mpst | 2.503 | 2.727 |
| Mptx1 | 2.165 | 5.081 |
| Mydgf | 2.403 | 2.589 |
| Ncoa4 | 2.587 | 3.552 |
| Ndufa4 | 2.681 | 1.914 |
| Ndufb4 | 10.847 | 2.049 |
| Ndufb5 | 2.979 | 2.367 |
| Nudt9 | 2.554 | 1.581 |
| Pabpc5 | 5.555 | 1.987 |
| Pitrm1 | 3.527 | 5.816 |
| Ppa2 | 2.437 | 1.777 |
| Psma3 | 2.232 | 1.692 |
| Psmd11 | 3.753 | 1.529 |
| Qdpr | 2.235 | 3.659 |
| Rab7 | 2.544 | 2.430 |
| Rack1 | 2.011 | 2.554 |
| Rap1b | 2.787 | 4.125 |
| Rbp2 | 2.056 | 2.677 |
| Rpl10a | 3.412 | 1.591 |
| Rpl17 | 2.154 | 2.040 |
| Rpl30 | 3.317 | 3.680 |
| Rplp1 | 2.074 | 1.857 |
| S100a11 | 2.032 | 1.929 |
| Smarce1 | 3.517 | 2.895 |
| Sult1a1 | 6.108 | 3.374 |
| Sumo2 | 7.113 | 2.997 |
| Tsta3 | 4.119 | 2.629 |
| Ugdh | 4.980 | 2.201 |
| Ugp2 | 2.834 | 2.939 |
| Ugt1a6b | 1.952 | 2.840 |
| Ugt2b34 | 5.825 | 5.698 |
| Vdac2 | 2.771 | 1.880 |
| Vdac3 | 3.515 | 3.891 |
| Wdr1 | 2.216 | 2.090 |

TABLE S5
Unique proteins in Msh2-HET gene (proteomic) signature.

| | Msh2-HET Lgr5^{EGFP+} | Msh2-HET Lgr5^{EGFP+} vs |
|---|---|---|
| Gene | vs Lgr5^{EGFP-} ratio | Msh2-WT Lgr5^{EGFP+} ratio |
| Acad9 | 2.706 | 1.411 |
| Actbl2 | 2.287 | 1.297 |
| Adal | 5.915 | 1.475 |
| Akr1c13 | 1.605 | 1.179 |
| Aldoart1 | 2.820 | 1.462 |
| Anp32a | 4.283 | 1.266 |
| Atad3 | 1.711 | 1.294 |
| Bag1 | 2.864 | 2.625 |
| Banf1 | 1.782 | 1.221 |
| Camk1d | 3.337 | 1.463 |
| Cbx1 | 3.732 | 1.300 |
| Copg1 | 2.292 | 1.257 |
| Csnk1a1 | 4.328 | 4.840 |
| Ddx17 | 4.846 | 2.193 |
| Dynlrb1 | 1.999 | 2.015 |
| Eif3c | 1.726 | 1.270 |
| Fasn | 2.125 | 1.705 |
| Gmps | 2.407 | 1.319 |
| Grb2 | 3.547 | 1.237 |
| H2afy | 10.227 | 4.067 |
| Hnrnpa0 | 1.857 | 1.211 |
| Hnrnph2 | 1.768 | 1.258 |
| Hsd17b10 | 2.114 | 1.181 |
| Ilf3 | 3.316 | 1.294 |
| Lsm14a | 1.628 | 1.213 |
| Mbnl1 | 3.264 | 1.430 |
| Mycbp | 1.645 | 1.615 |
| Nudt21 | 6.145 | 1.566 |
| Nudt5 | 2.359 | 1.723 |
| Pabpc4 | 1.619 | 1.500 |
| Pafah1b3 | 2.886 | 1.739 |
| Pak2 | 2.058 | 1.375 |
| Parp1 | 2.438 | 2.538 |
| Pfdn2 | 2.057 | 1.359 |
| Pgm2 | 1.641 | 1.260 |
| Pitrm1 | 3.323 | 2.041 |
| Prpf19 | 2.594 | 1.920 |
| Psmc4 | 1.698 | 1.529 |
| Psmd8 | 2.822 | 1.229 |
| Rnps1 | 3.027 | 1.505 |
| Rpa3 | 2.713 | 2.625 |
| Rpl13a | 1.639 | 1.503 |
| Rpl13a-ps1 | 1.760 | 1.484 |
| Rpl23a | 2.242 | 1.184 |
| Rpl38 | 2.167 | 1.398 |
| Rps18 | 3.194 | 1.420 |
| Rsl1d1 | 2.773 | 1.263 |
| Sec13 | 1.603 | 1.741 |
| Sf3b1 | 2.393 | 1.468 |
| Srprb | 2.163 | 1.538 |
| Srsf3 | 1.859 | 1.278 |
| Tial1 | 3.654 | 2.477 |

TABLE S6
Clinical characteristics of patient samples used for validation for MMR insufficiency
and MMR deficiency gene signature. Female, F; Male, M; TA, Tubular Adenoma; Adenocarcinoma,
AC. Germline mutations in Adenomatous Polyposis Coli, APC, constitute for patients with
familial adenomatous polyposis (FAP) and those with mutationsd in mismatch repair genes,
MLH1, MSH2, MSH6, and PMS2 constitute for Lynch Syndrome with the exception of patient
LS27 that has a diagnosis of clinical LS without a germline mutation detected. Hx, History;
TA, Tubular adenoma; AC, adenocarcinoma; ND, not detected.

| Sample | | | | | Germline Mutation | |
|---|---|---|---|---|---|---|
| ID | Gender | Age | Race | Cancer Hx | Gene | DNA mutation |
| FAP 1 | F | 76 | Caucasian | No | APC | codon 77 del 4bp |
| FAP 2 | M | 34 | Caucasian | No | APC | no information |
| FAP 3 | F | 58 | Caucasian | ampulla | APC | Codon 151 del 1bp |
| | | | | de vater | | |
| FAP 8 | M | 33 | Caucasian | No | APC | 3810T > A |
| FAP 4 | M | 25 | Unknown | No | APC | 1957A > C |
| FAP 5 | M | 57 | Caucasian | No | APC | no information |
| FAP 6 | M | 35 | Caucasian | No | APC | codon 626 insert |
| | | | | | | 1 base pair |
| FAP 7 | F | 42 | Caucasian | Small Bowel | APC | Del exon 14 |
| FAP 9 | M | 42 | Caucasian | Liver/Desmoid | APC | Codon 208 |
| | | | | | | CAG to TAG |
| FAP 10 | F | 40 | Other | No | APC | Del Exon 8-9 |
| FAP 11 | M | 37 | Caucasian | No | APC | 1658G > A |
| FAP 12 | M | 65 | Caucasian | No | APC | C515G |
| FAP 13 | F | 25 | African | Desmoid tumor | APC | no information |
| | | | American | | | |
| FAP 14 | F | 28 | Caucasian | Endometrial | APC | 847C > T |
| FAP 15 | F | 25 | Caucasian | No | APC | 3810T > A |
| FAP 16 | M | 45 | Other | Small bowel | APC | Del exon 4 |
| FAP 17 | F | 23 | Caucasian | Medulloblastoma | APC | 3441insA |
| LS1 | M | 73 | Caucasian | Colon/Lung/ | MSH2 | 1906G > C |
| | | | | Melanoma | | |
| LS2 | M | 35 | Caucasian | Rectum | MLH1 | 207 + 2T > C |
| LS3 | M | 50 | other | Colon | MLH1 | 1790delGGins9 |
| LS4 | M | 56 | Caucasian | Colon | MLH1 | del Exon 15 |
| LS5 | F | 36 | Caucasian | | MLH1 | |
| LS6 | F | 65 | Caucasian | Colon | MSH2 | 2634 + 5G > C |
| LS7 | M | 68 | Caucasian | Prostate | MSH6 | 3939_3957dup |
| LS8 | F | 72 | Caucasian | Promylocytic | MSH6 | 3439 − 2A > G |
| | | | | Leukemia | | |
| LS9 | M | 52 | Caucasian | Colon | MSH6 | c.3744_3773del130 |
| LS10 | F | 46 | Caucasian | Endometrial | MSH2 | 687delA |
| LS11 | M | 52 | Caucasian | Colon | PMS2 | del exon 4 |
| LS12 | F | 37 | Caucasian | Colon | MSH2 | 1034G < A |
| LS13 | F | 53 | Caucasian | | MSH2 | IVS10 + 1G > A |
| LS14 | M | 58 | Caucasian | | MSH2 | del Exon 1-6 |
| LS15 | M | 76 | Caucasian | Colon | MSH2 | 1216C > T |
| LS16 | F | 66 | Caucasian | Uterus | MSH6 | 3238_3239delCT |
| LS17 | F | 43 | Other | Colon | MSH6 | 2645_2653delTTAAGTCTA |
| LS18 | F | 63 | Caucasian | Endometrial | MSH6 | 3699-3702delAGAA |
| LS19 | F | 62 | Caucasian | Endometrial | MSH6 | 3850ins4 |
| LS20 | F | 55 | Caucasian | Sebaceous | MSH6 | 3699del4 |
| | | | | carc vulva | | |
| LS21 | M | 47 | Caucasian | | MSH2 | IVS5 + 3A > T |
| LS22 | M | 28 | Caucasian | | MSH2 | IVS5 + 3A > T |
| LS23 | F | 68 | Caucasian | Colon/Uterus | MSH2 | del exon 1-6 |
| | | | | and ureter | | |
| LS24 | F | 77 | Caucasian | Colon/ | PMS2 | 1927C > T |
| | | | | Endometrial | | |
| LS25 | M | 48 | Other | Colon | MLH1 | 1790_1791delGGinsATCTGGACC |
| LS26 | M | 53 | Caucasian | Colon | MSH2 | IVS5 + 3A > T |
| LS27 | F | 42 | Caucasian | Colon | ND | ND |

| | | Sample information | | |
|---|---|---|---|---|
| Sample | Germline Mutation | Normal | | |
| ID | Protein mutation | Mucosa | TA | AC |
| FAP 1 | | H_G86 | H_G85 | |
| FAP 2 | no information | H_G93 | H_G87 | |
| FAP 3 | | H_G95 | H_G94 | |
| FAP 8 | 1270x | H_G54 | H_G53 | |
| FAP 4 | R653R | H_G106 | H_G102 | |
| FAP 5 | no information | H_G110 | H_G108 | |
| FAP 6 | | H_G1 | H_G2 | |
| FAP 7 | | H_G5 | H_G4 | |
| FAP 9 | | H_G16 | H_G13 | |
| FAP 10 | | H_G26 | H_G25 | |
| FAP 11 | Trp553* | H_G31 | H_G29 | |
| FAP 12 | Y159* | H_G40 | H_G39 | |
| FAP 13 | no information | H_G49 | H_G47 | |
| FAP 14 | R283X | H_G60 | H_G67 | |
| FAP 15 | 1270x | H_G62 | H_G61 | |
| FAP 16 | | H_G121 | H_G119 | |
| FAP 17 | Y1147X | H_G84 | H_G79 | |
| LS1 | R406X | ND19 | ND20 | |
| LS2 | | ND21 | | ND22 |
| LS3 | 611Stop | ND23 | | ND24 |
| LS4 | | ND27 | | ND28 |
| LS5 | Q86* | ND36 | | |
| LS6 | | ND39 | | |
| LS7 | A1320Sfs*5 | ND41 | | |
| LS8 | | ND43 | | |
| LS9 | | EBL2 | | |
| LS10 | Ala230Leufs*16 | EBL4 | | |
| LS11 | | EBL6 | | |
| LS12 | trp345ter | EBL8 | | |
| LS13 | | EBL10 | EBL9 | |
| LS14 | | EBL12 | EBL11 | |
| LS15 | R406X | EBL14 | | |
| LS16 | L1080VfsX12 | EBL19 | | |
| LS17 | F822X | EBL21 | EBL20 | NA12 |
| LS18 | | ND34/EBL24 | | |
| LS19 | | EBL27 | | |
| LS20 | | EVL30 | | |
| LS21 | | EVL32 | | |
| LS22 | | EVL34 | | |
| LS23 | | EVL35/EVL42 | NA31 | NA30 |
| LS24 | Q643* | EVL39 | | |
| LS25 | W597YfsX15 | EVL41 | | NA20 |
| LS26 | 942 + 3A > T | | NA26 | NA27/NA21 |
| LS27 | ND | ND29 | | |

TABLE S7
List of primers used in this example.

| Gene Name | Forward | Reverse |
|---|---|---|
| Lgr5 | ACCTGTGGCTAGATGACAATGC | TCCAAAGGCGTAGTCTGCTAT |
| Ascl2 | AAGCACACCTTGACTGGTACG | AAGTGGACGTTTGCACCTTCA |
| Olfm4 | GGACAGGACTCAGGTTGCGG | CCAGCGTGTGAAGAAAGAGGA |
| Alpi | GGCCATCTAGGACCGGAGA | TGTCCACGTTGTATGTCTTGG |
| Krt20 | TTCAGTCGTCAAAGTTTTCACCG | TCCTATACAGCGAGCCACTCA |
| Gapdh | AGGTCGGTGTGAACGGATTTG | TGTAGACCATGTAGTTGAGGTCA |
| Spp1 | AGAGCGGTGAGTCTAAGGAGT | TGCCCTTTCCGTTGTTGTCC |
| Nr1h5 | ACCTATGTTGCTACGTCTGATGG | GGGTGGTTCTTGGAAATCTGTAT |
| Ahnak | GCTCCTGAGGTGAAAGGTGAT | TGATCTTGGGACCTGTCAAGG |
| Nlrp9b | CCAAGGGTGATAACTATGCAAGG | TCAGCTACTTCTATGCCTCTCTG |
| Muc5ac | GTGGTTTGACACTGACTTCCC | CTCCTCTCGGTGACAGAGTCT |
| SPP1 - proximal | CCGAAAGAAACAAAAATCCATTCT | CAGCATCCAGGAAGAGCACTT |
| promoter | | |
| SPP1 - 3′UTR | CTTAGGCTCCCAGCATACTTGCT | CACCATCAATCCAGCCATCA |

TABLE S8
List of antibodies used in this example

| Antibody | Source | Catalog # |
|---|---|---|
| AF488-conjugated Rabbit polyclonal Anti-GFP (used at 1:100) | Invitrogen | A-21311 |
| Goat Polyclonal Anti-Osteopontin (used at 5 μg/ml) | R&D Systems | AF808 |
| Mouse monoclonal anti-LGR5 (used at 1:100) | Origene | TA503316 |
| Rabbit polyclonal anti-Osteopontin (used at 1:50) | Abcam | ab8448 |
| Rat anti-mouse CD45, PerCP/Cy5.5 conjugated clone 30-F11 (Used | FisherScientific | BDB550994 |
| at 0.2 μg/ml) | | |
| Rat anti-mouse CD326 (EpCam), PE conjugated clone G8.8 (Used at | FisherScientific | BDB563477 |
| 0.2 μg/ml) | | |
| NL557-conjugated Northern Lights Donkey polyclonal Anti-Goat IgG | R&D Systems | NL001 |
| FITC-conjugated Rabbit monoclonal Anti-Mouse IgG (used at 1:200) | Abcam | ab6724 |
| TRITC-conjugated Goat Polyclonal Anti-Rabbit IgG (used at 1:200) | Invitrogen | A16101 |
| Histone H3K4me Rabbit Polyclonal | Active Motif | 39159 |
| H3K27me3 recombinant Rabbit Polyclonal | Diagenode | C1541095 |

TABLE S9
Clinical characteristics of LS patient samples used in IHC. Female, F; Male,
M; Normal Mucosa, NM; Tubular Adenoma, TA; Tubulovillous Adenoma, TVA;
Adenocarcinoma, AC. Pathogenic germline mutations in mismatch repair genes
MLH1, MSH2, MSH6, and PMS2 are diagnostic of Lynch Syndrome (LS).

| Patient | | | | | |
|---|---|---|---|---|---|
| ID | Tissue | Gender | Age | Gene | Mutation |
| Patient 1 | NM, AC | M | 69 | MLH1 | del Exon 16 |
| Patient 2 | NM, AC | F | 41 | MSH6 | 2645_2653delTTAAGTCTA |
| Patient 3 | NM, TA, AC | M | 49 | MSH2 | 942 + 3A > T |
| Patient 4 | NM, TA | M | 27 | MSH2 | 2063T > G |
| Patient 5 | NM, TVA | F | 77 | PMS2 | 1927C > T |
| Patient 6 | NM, TVA | F | 43 | MSH2 | del Exon 3_7 |

### XI. Example 2: SPP1 Immunohistochemistry in Tissue Sections Demonstrate Detection of Tumors

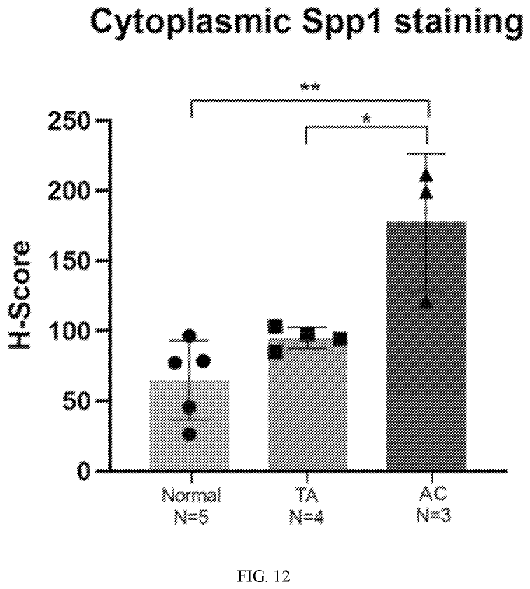

Methods: Immunohistochemistry (IHC) staining for SPP1 was performed in FFPE tissue sections. Tissue sections were cut at 5μm and submitted to the MDACC Research Histology, Pathology, and Imaging Core (RHPI) in Smithville, TX. Osteopontin (OPN) antibody (Assay Designs, catalog # 905-629) was used according to manufacturer's recommendations. The stained slides were scanned with Aperio ScanScope® CS at 20X magnification. Using Aperio eSlideManager, selected regions of interest (ROIs) were manually defined and annotated to include colonic normal mucosa, tubular adenoma (TA), and adenocarcinoma (AC). H-score was obtained according to intensity as 0, negative; 1+, weak; 2+, moderate; 3+, strong. The percentage of positively stained cells (0 to 100) was multiplied by the staining intensity score (0/1/2/3), thus yielding scores from 0 to 300 (Gothlin Eremo A, et al., Sci Rep. 2020;10(1):1451. doi: 10.1038/s41598-020-58323-w. PubMed PMID: 31996744; PubMed Central PMCID: PMCPMC6989629). Results: The inventors examined the levels of SPP1 in Lynch syndrome tissue specimens (see table below) representing sequential steps of colorectal carcinogenesis: normal mucosa (n=5), pre-cancer (n=4, tubular adenoma), and tumors (n=3, adenocarcinomas). Using one-way ANOVA, the H-score was significantly higher among adenocarcinomas compared to tubular adenomas and normal mucosa samples (177.3 vs 95.08, P-value 0.0149; 177.3 vs 64.76, P-value 0.016, respectively, FIG. 12).

TABLE
Clinical characteristics of LS patient samples used in IHC mediated staining.
Female, F; Male, M; Normal Mucosa, NM; Tubular Adenoma, TA; Adenocarcinoma,
AC. Low-grade dysplasia, LGD; High-Grade Dysplasia, HGD. Germline mutations
in mismatch repair genes, MLH1, MSH2, and MSH6 constitute for Lynch Syndrome
(LS) including Lynch Like Syndrome (LLS).

| Patient ID | Tissue | Gender | Age | Gene | Mutation |
|---|---|---|---|---|---|
| Patient 1 | NM, AC | F | 43 | MSH6 | 2645_2653delTTAAGTCTA |
| Patient 2 | NM, TA | F | 56 | MSH2 | 1295T > C |
| Patient 3 | NM, AC | M | 53 | MSH2 | 942 + 3A > T |
| Patient 4 | NM, TA | M | 68 | MSH6 | 3939_3957dup |
| Patient 5 | NM, TA | M | 73 | MSH2 | 1906G > C |
| | LGD, TA | | | | |
| | HGD | | | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Lynch H T, Snyder C L, Shaw T G, et al. Milestones of Lynch syndrome: 1895-2015. Nat Rev Cancer 2015;15:181-94.
2. Win A K, Jenkins M A, Dowty J G, et al. Prevalence and Penetrance of Major Genes and Polygenes for Colorectal Cancer. Cancer Epidemiol Biomarkers Prey 2017;26:404-412.
3. Vilar E, Gruber S B. Microsatellite instability in colorectal cancer-the stable evidence. Nat Rev Clin Oncol 2010;7:153-62.
4. Fearon ER. Molecular genetics of colorectal cancer. Annu Rev Pathol 2011;6:479-507.
5. Bonadona V, Bonaiti B, Olschwang S, et al. Cancer risks associated with germline mutations in MLH1, MSH2, and MSH6 genes in Lynch syndrome. JAMA 2011;305:2304-10.
6. Stoffel E, Mukherjee B, Raymond V M, et al. Calculation of risk of colorectal and endometrial cancer among patients with Lynch syndrome. Gastroenterology 2009;137:1621-7.
7. Moller P, Seppala T, Bernstein I, et al. Cancer incidence and survival in Lynch syndrome patients receiving colonoscopic and gynaecological surveillance: first report from the prospective Lynch syndrome database. Gut 2017;66:464-472.
8. Schmeler K M, Lynch H T, Chen L M, et al. Prophylactic surgery to reduce the risk of gynecologic cancers in the Lynch syndrome. N Engl J Med 2006;354:261-9.
9. van Leerdam M E, Roos V H, van Hooft J E, et al. Endoscopic management of Lynch syndrome and of familial risk of colorectal cancer: European Society of Gastrointestinal Endoscopy (ES GE) Guideline. Endoscopy 2019;51:1082-1093.
10. Spira A, Yurgelun M B, Alexandrov L, et al. Precancer Atlas to Drive Precision Prevention Trials. Cancer Res 2017;77:1510-1541.
11. Barker N. Adult intestinal stem cells: critical drivers of epithelial homeostasis and regeneration. Nat Rev Mol Cell Biol 2014;15:19-33.
12. Sato T, Clevers H. Growing self-organizing mini-guts from a single intestinal stem cell: mechanism and applications. Science 2013;340:1190-4.
13. Vermeulen L, Snippert HJ . Stem cell dynamics in homeostasis and cancer of the intestine. Nat Rev Cancer 2014;14:468-80.
14. Visvader J E. Cells of origin in cancer. Nature 2011;469:314-22.
15. Barker N, Ridgway R A, van Es J H, et al. Crypt stem cells as the cells-of-origin of intestinal cancer. Nature 2009;457:608-11.
16. Barker N, van Es J H, Kuipers J, et al. Identification of stem cells in small intestine and colon by marker gene Lgr5. Nature 2007;449:1003-7.
17. Munoz J, Stange D E, Schepers A G, et al. The Lgr5 intestinal stem cell signature: robust expression of proposed quiescent ‘+4’ cell markers. EMBO J 2012;31:3079-91.
18. de Wind N, Dekker M, Berns A, et al. Inactivation of the mouse Msh2 gene results in mismatch repair deficiency, methylation tolerance, hyperrecombination, and predisposition to cancer. Cell 1995;82:321-30.
19. Vaish M. Mismatch repair deficiencies transforming stem cells into cancer stem cells and therapeutic implications. Mol Cancer 2007;6:26.
20. Kucherlapati M H, Lee K, Nguyen A A, et al. An Msh2 conditional knockout mouse for studying intestinal cancer and testing anticancer agents. Gastroenterology 2010;138:993-1002 el.
21. Chang K, Taggart M W, Reyes-Uribe L, et al. Immune Profiling of Premalignant Lesions in Patients With Lynch Syndrome. JAMA Oncol 2018.
22. Reyes-Uribe L, Wu W, Gelincik O, et al. Naproxen chemoprevention promotes immune activation in Lynch syndrome colorectal mucosa. Gut 2020.
23. Costales-Carrera A, Fernandez-Banal A, Bustamante-Madrid P, et al. Comparative Study of Organoids from Patient-Derived Normal and Tumor Colon and Rectal Tissue. Cancers (Basel) 2020;12.
24. Tetteh P W, Kretzschmar K, Begthel H, et al. Generation of an inducible colon-specific Cre enzyme mouse line for colon cancer research. Proc Natl Acad Sci USA 2016;113:11859-11864.
25. Kumar R, Raman R, Kotapalli V, et al. Ca(2+)/nuclear factor of activated T cells signaling is enriched in early-onset rectal tumors devoid of canonical Wnt activation. J Mol Med (Berl) 2018;96:135-146.
26. de The H. Differentiation therapy revisited. Nat Rev Cancer 2018;18:117-127.
27. Fischer M M, Yeung V P, Cattaruzza F, et al. RSPO3 antagonism inhibits growth and tumorigenicity in colorectal tumors harboring common Wnt pathway mutations. Sci Rep 2017;7:15270.
28. Storm E E, Durinck S, de Sousa e Melo F, et al. Targeting PTPRK-RSPO3 colon tumours promotes differentiation and loss of stem-cell function. Nature 2016;529:97-100.
29. Zhang W, Tong D, Liu F, et al. RPS7 inhibits colorectal cancer growth via decreasing HIF-1alpha-mediated glycolysis. Oncotarget 2016;7:5800-14.
30. Zhou X, Hao Q, Liao J M, et al. Ribosomal protein S14 negatively regulates c-Myc activity. J Biol Chem 2013;288:21793-801.
31. Friedmann-Morvinski D, Verma I M. Dedifferentiation and reprogramming: origins of cancer stem cells. EMBO Rep 2014;15:244-53.
32. Cerretelli G, Ager A, Arends M J, et al. Molecular pathology of Lynch syndrome. J Pathol 2020;250:518-531.
33. Huang E H, Hynes M J, Zhang T, et al. Aldehyde dehydrogenase 1 is a marker for normal and malignant human colonic stem cells (SC) and tracks SC overpopulation during colon tumorigenesis. Cancer Res 2009;69:3382-9.
34. Kim J H, Skates S J, Uede T, et al. Osteopontin as a potential diagnostic biomarker for ovarian cancer. JAMA 2002;287:1671-9.
35. Rodrigues L R, Teixeira J A, Schmitt F L, et al. The role of osteopontin in tumor progression and metastasis in breast cancer. Cancer Epidemiol Biomarkers Prey 2007;16:1087-97.
36. Rangaswami H, Bulbule A, Kundu G C. Osteopontin: role in cell signaling and cancer progression. Trends Cell Biol 2006;16:79-87.
37. Cheng Y, Wen G, Sun Y, et al. Osteopontin Promotes Colorectal Cancer Cell Invasion and the Stem Cell-Like Properties through the PI3K-AKT-GSK/3beta-beta/Catenin Pathway. Med Sci Monit 2019;25:3014-3025.
38. Ng L, Wan T, Chow A, et al. Osteopontin Overexpression Induced Tumor Progression and Chemoresistance to Oxaliplatin through Induction of Stem-Like Properties in Human Colorectal Cancer. Stem Cells Int 2015;2015:247892.
39. Zhu Y, Yang J, Xu D, et al. Disruption of tumour-associated macrophage trafficking by the osteopontin-induced colony-stimulating factor-1 signalling sensitises hepatocellular carcinoma to anti-PD-Ll blockade. Gut 2019;68:1653-1666.
40. Chen P, Zhao D, Li J, et al. Symbiotic Macrophage-Glioma Cell Interactions Reveal Synthetic Lethality in PTEN-Null Glioma. Cancer Cell 2019;35:868-884 e6.
41. Klement JD, Paschall AV, Redd PS, et al. An osteopontin/CD44 immune checkpoint controls CD8+ T cell activation and tumor immune evasion. J Clin Invest 2018;128:5549-5560.
42. Shurin MR. Osteopontin controls immunosuppression in the tumor microenvironment. J Clin Invest 2018;128:5209-5212.
43. Akcora D, Huynh D, Lightowler S, et al. The CSF-1 receptor fashions the intestinal stem cell niche. Stem Cell Res 2013;10:203-12.

44. Wang Y, Han G, Wang K, et al. Tumor-derived GM-CSF promotes inflammatory colon carcinogenesis via stimulating epithelial release of VEGF. Cancer Res 2014;74:716-26.
45. Lu R, Markowetz F, Unwin RD, et al. Systems-level dynamic analyses of fate change in murine embryonic stem cells. Nature 2009;462:358-62.
46. Schwanhausser B, Busse D, Li N, et al. Global quantification of mammalian gene expression control. Nature 2011;473:337-42.

What is claimed is:

1. A method for treating a subject for colorectal cancer, the method comprising treating the subject for colorectal cancer after the expression level of one or more biomarker genes from Table 1, Table 2, Table 3, Table 4, Table 5, Table S2, Table S3, Table S4, Table S5, FIG. 4A, and/or FIG. 4B has been determined in a sample from the subject.

2. The method of claim 1, wherein at least 5 biomarkers from Table 1, Table 2, Table 3, Table 4, Table 5, Table S2, Table S3, Table S4, and/or Table S5 has been determined.

3. The method of claim 1 or 2, wherein at least 5 biomarkers from Table 2 were determined in a sample from the subject.

4. The method of any one of claims 1-3, wherein at least 6 biomarkers from Table 2 were determined in a sample from the subject.

5. The method of any one of claims 1-4, wherein at least 7 biomarkers from Table 2 were determined in a sample from the subject.

6. The method of any one of claims 1-5, wherein at least 8 biomarkers from Table 2 were determined in a sample from the subject.

7. The method of any one of claims 1-6, wherein at least 9 biomarkers from Table 2 were determined in a sample from the subject.

8. The method of any one of claims 1-7, wherein at least Spp1 was determined in a sample from the subject.

9. The method any one of claims 1-8, wherein at least Mup22 was determined in a sample from the subject.

10. The method any one of claims 1-9, wherein at least S1c26a9 was determined in a sample from the subject.

11. The method any one of claims 1-10, wherein at least Muc6 was determined in a sample from the subject.

12. The method any one of claims 1-11, wherein at least Ugt8a was determined in a sample from the subject.

13. The method any one of claims 1-12, wherein at least Meg3 was determined in a sample from the subject.

14. The method any one of claims 1-13, wherein at least P2ry4 was determined in a sample from the subject.

15. The method any one of claims 1-14, wherein at least Defa5 was determined in a sample from the subject.

16. The method any one of claims 1-15, wherein at least Gm49320 was determined in a sample from the subject.

17. The method of any one of claims 1-16, wherein the method further comprises calculating a prognosis score.

18. The method of claim 17, wherein the prognosis score comprises an H-score.

19. The method of any one of claims 1-18, wherein the expression level of the biomarker was determined by immunofluorescence.

20. The method of claim 19, wherein the expression level of the biomarker was determined by immunohistochemistry staining for the biomarker in tissue sections.

21. The method of any one of claims 18-20, wherein the subject is determined to have a H-score of greater than 100.

22. The method of any one of claims 1-21, wherein the subject has been determined to have Lynch Syndrome.

23. The method of any one of claims 1-16, wherein the subject has not been diagnosed with Lynch Syndrome.

24. The method of any one of claims 1-23, wherein the subject has not been diagnosed with and/or has not been treated for colorectal cancer.

25. The method of any one of claims 1-24, wherein the subject is treated for stage I or stage II colorectal cancer.

26. The method of any one of claims 1-25, wherein the colorectal cancer comprises mismatch repair deficient colorectal cancer (MMR-d).

27. The method of any one of claims 1-26, wherein the expression level is normalized.

28. The method of any one of claims 1-27, wherein the sample from the subject is a sample from a primary colorectal cancer tumor.

29. The method of any one of claims 1-27, wherein the sample from the subject is a sample from a biopsy of colorectal tissues.

30. The method of any one of claims 1-29, wherein the sample from the subject is a sample of colon mucosa or a culture of cells derived from the colon mucosa of the subject.

31. The method of claim 30, wherein the sample comprises an organoid derived from the colon mucosa of the subject.

32. The method of any one of claims 1-31, wherein the expression levels of the one or more biomarkers in the subject was determined to be i) increased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

33. The method of any one of claims 1-32, wherein the expression levels of the one or more biomarkers in the subject was determined to be i) decreased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

34. The method of any one of claims 1-33, wherein the treatment comprises one or more of surgery, partial colectomy, surgical removal of lymph nodes, radiation, targeting therapy, adjuvant chemotherapy, and neo adjuvant chemotherapy.

35. The method of claim 34, wherein the treatment excludes one or more of surgery, partial colectomy, surgical removal of lymph nodes, radiation, targeting therapy, adjuvant chemotherapy, and neo-adjuvant chemotherapy.

36. The method of claim 35, wherein the treatment excludes chemotherapy, adjuvant chemotherapy, or neo-adjuvant chemotherapy.

37. The method of claim 36, wherein the chemotherapy comprises one or more of 5-FU, leucovorin, oxaliplatin, capecitabine, and irinotecan.

38. The method of any one of claims 34-37, wherein the targeted therapy comprises one or more of bevacizumab, ziv-aflibercept, ramucirumab, cetuximab, and panitumumab.

39. The method of any one of claims 34-37, wherein the targeted therapy excludes one or more of bevacizumab, ziv-aflibercept, ramucirumab, cetuximab, and panitumumab.

40. The method of any one of claims 1-39, wherein the treatment comprises one or more of regorafenib, trifluridine, and tipiracil.

41. The method of any one of claims 1-39, wherein the treatment excludes one or more of regorafenib, trifluridine, and tipiracil.

42. The method of any one of claims 1-41, wherein the expression level of the biomarker has been determined in the subject by determining the amount of protein produced from the biomarker gene.

43. The method of any one of claims 1-41, wherein the expression level of the biomarker has been determined in the subject by determining the amount of mRNA produced from the biomarker gene.

44. The method of any of claims 1-43, wherein the subject has undergone surgery to resect all or part of the cancer.

45. The method of any one of claims 1-44, wherein the subject has not undergone surgical resection of the tumor.

46. The method of any of claims 1-45, wherein the level of expression of one of more of the biomarker genes was determined pre-operative and/or post-operative.

47. The method of any one of claims 32-46, wherein low risk is indicative of a subject with a low risk for distant metastasis and good overall survival (OS) rate, and high risk is indicative of a subject with a high risk for distant metastasis and poor overall survival (OS) rate.

48. The method of any one of claims 1-47, wherein the method further comprises evaluating the sample from the subject for MMR-d and/or microsatellite instability (MSI).

49. The method of any one of claims 1-48, wherein the MMR-d and/or MSI has been evaluated in the subject.

50. The method of claim 48 or 49, wherein evaluating the sample from the subject for MMR-d comprises determining the level of expression of one or more of MLH1, MSH2, MSH6, PMS2, and EPCAM.

51. The method of any one of claims 48-50, wherein the subject has been determined to be MMR-d and/or MSI+.

52. The method of any one of claims 1-51, wherein the cancer comprises stage 0, I, II, III, or IV cancer.

53. The method of any one of claims 1-52, wherein the cancer excludes stage 0, I, II, III, or IV cancer.

54. A method for evaluating a subject comprising measuring the level of expression of one or more biomarkers of Table 1, Table 2, Table 3, Table 4, Table 5, Table S2, Table S3, Table S4, Table S5, FIG. 4A, and/or FIG. 4B in a sample from the subject.

55. The method of claim 54, wherein at least 5 biomarkers from Table 1, Table 2, Table 3, Table 4, Table 5, Table S2, Table S3, Table S4, and/or Table S5 are measured.

56. The method of claim 54 or 55, wherein at least 5 biomarkers from Table 2 are measured in the sample from the subject.

57. The method of any one of claims 54-56, wherein at least Spp1 is measured in the sample from the subject.

58. The method any one of claims 54-57, wherein at least Mup22 is measured in the sample from the subject.

59. The method any one of claims 54-58, wherein at least S1c26a9 is measured in the sample from the subject.

60. The method any one of claims 54-59, wherein at least Muc6 is measured in the sample from the subject.

61. The method any one of claims 54-60, wherein at least Ugt8a is measured in the sample from the subject.

62. The method any one of claims 54-61, wherein at least Meg3 is measured in the sample from the subject.

63. The method any one of claims 54-62, wherein at least P2ry4 is measured in the sample from the subject.

64. The method any one of claims 54-63, wherein at least Defa5 is measured in the sample from the subject.

65. The method any one of claims 54-64, wherein at least Gm49320 is measured in the sample from the subject.

66. The method of any one of claims 54-65, wherein the subject has been determined to have Lynch Syndrome.

67. The method of any one of claims 54-65, wherein the subject has not been diagnosed with Lynch Syndrome.

68. The method of any one of claims 54-67, wherein the subject has not been diagnosed with and/or has not been treated for colorectal cancer.

69. The method of any one of claims 54-68, wherein the method further comprises treating the subject.

70. The method of claim 69, wherein the subject is treated for stage I or stage II colorectal cancer.

71. The method of any one of claims 68-70, wherein the colorectal cancer comprises mismatch repair deficient colorectal cancer (MMR-d).

72. The method of any one of claims 54-71, wherein the expression level is normalized.

73. The method of any one of claims 54-72, wherein the sample from the subject is a sample from a primary colorectal cancer tumor.

74. The method of any one of claims 54-72, wherein the sample from the subject is a sample from a biopsy of colorectal tissues.

75. The method of any one of claims 54-74, wherein the sample from the subject is a sample of colon mucosa or a culture of cells derived from the colon mucosa of the subject.

76. The method of claim 75, wherein the sample comprises an organoid derived from the colon mucosa of the subject.

77. The method of any one of claims 54-76, wherein the sample from the subject comprises a tissue sample.

78. The method of any one of claims 54-77, wherein measuring the level of expression of the biomarker comprises immunological detection of the biomarker protein.

79. The method of claim 78, wherein the method comprises immunohistochemistry staining for the biomarker in tissue sections.

80. The method of any one of claims 54-79, wherein the method further comprises calculating a prognosis score.

81. The method of claim 80, wherein the score comprises a H-score.

82. The method of any one of claims 54-81, wherein the expression levels of the one or more biomarkers in the subject is i) increased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

83. The method of any one of claims 54-82, wherein the expression levels of the one or more biomarkers in the subject is i) decreased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

84. The method of any one of claims 69-83, wherein the treatment comprises one or more of surgery, partial colectomy, surgical removal of lymph nodes, radiation, targeting therapy, adjuvant chemotherapy, and neo adjuvant chemotherapy.

85. The method of claim 84, wherein the treatment excludes one or more of surgery, partial colectomy, surgical removal of lymph nodes, radiation, targeting therapy, adjuvant chemotherapy, and neo-adjuvant chemotherapy.

86. The method of claim 85, wherein the treatment excludes chemotherapy, adjuvant chemotherapy, or neo-adjuvant chemotherapy.

87. The method of claim 86, wherein the chemotherapy comprises one or more of 5-FU, leucovorin, oxaliplatin, capecitabine, and irinotecan.

88. The method of any one of claims 84-87, wherein the targeted therapy comprises one or more of bevacizumab, ziv-aflibercept, ramucirumab, cetuximab, and panitumumab.

89. The method of any one of claims 84-87, wherein the targeted therapy excludes one or more of bevacizumab, ziv-aflibercept, ramucirumab, cetuximab, and panitumumab.

90. The method of any one of claims 69-89, wherein the treatment comprises one or more of regorafenib, trifluridine, and tipiracil.

91. The method of any one of claims 69-89, wherein the treatment excludes one or more of regorafenib, trifluridine, and tipiracil.

92. The method of any one of claims 54-91, wherein measuring the level of expression of one or more biomarkers comprises measuring the amount of protein produced from the biomarker gene.

93. The method of any one of claims 54-91, wherein measuring the level of expression of one or more biomarkers comprises measuring the amount of mRNA produced from the biomarker gene.

94. The method of any of claims 54-93, wherein the subject has undergone surgery to resect all or part of the cancer.

95. The method of any one of claims 54-94, wherein the subject has not undergone surgical resection of the tumor.

96. The method of any of claims 54-95, wherein the sample comprises a pre-operative and/or post-operative.

97. The method of any one of claims 82-96, wherein low risk is indicative of a subject with a low risk for distant metastasis and good overall survival (OS) rate, and high risk is indicative of a subject with a high risk for distant metastasis and poor overall survival (OS) rate.

98. The method of any one of claims 54-97, wherein the method further comprises evaluating the sample from the subject for MMR-d and/or microsatellite instability (MSI).

99. The method of any one of claims 54-98, wherein the sample is from a subject that has been evaluated for MMR-d and/or MSI.

100. The method of claim 98 or 99, wherein evaluating the sample from the subject for MMR-d comprises determining the level of expression of one or more of MLH1, MSH2, MSH6, PMS2, and EPCAM.

101. The method of any one of claims 98-100, wherein the sample is from a subject has been determined to be MMR-d and/or MSI+.

102. The method of any one of claims 54-101, wherein the cancer comprises stage 0, I, II, III, or IV cancer.

103. The method of any one of claims 54-102, wherein the cancer excludes stage 0, I, II, III, or IV cancer.

104. A method of prognosing and/or diagnosing a subject with colorectal cancer comprising
a) measuring the level of expression of one or more biomarker genes from Table 1, Table 2, Table 3, Table 4, Table 5, Table S2, Table S3, Table S4, Table S5, FIG. 4A, and/or FIG. 4B in a sample from the subject;
b) comparing the level(s) of expression to a control sample(s) or control level(s) of expression; and,
c) prognosing and/or diagnosing the subject based on the levels of measured expression.

105. The method of claim 104, wherein at least 5 biomarkers from from Table 1, Table 2, Table 3, Table 4, Table 5, Table S2, Table S3, Table S4, Table S5, FIG. 4A, and/or FIG. 4B are measured.

106. The method of claim 104 or 105, wherein at least 5 biomarkers from Table 2 are measured in the sample from the subject.

107. The method of any one of claims 104-106, wherein at least Spp 1 is measured in the sample from the subject.

108. The method any one of claims 104-107, wherein at least Mup22 is measured in the sample from the subject.

109. The method any one of claims 104-108, wherein at least S1c26a9 is measured in the sample from the subject.

110. The method any one of claims 104-109, wherein at least Muc6 is measured in the sample from the subject.

111. The method any one of claims 104-110, wherein at least Ugt8a is measured in the sample from the subject.

112. The method any one of claims 104-111, wherein at least Meg3 is measured in the sample from the subject.

113. The method any one of claims 104-112, wherein at least P2ry4 is measured in the sample from the subject.

114. The method any one of claims 104-113, wherein at least Defa5 is measured in the sample from the subject.

115. The method any one of claims 104-114, wherein at least Gm49320 is measured in the sample from the subject.

116. The method of any one of claims 1-16, wherein the method further comprises calculating a prognosis score.

117. The method of claim 17, wherein the prognosis score comprises an H-score.

118. The method of any one of claims 1-18, wherein the expression level of the biomarker is determined by immunofluorescence.

119. The method of claim 19, wherein the expression level of the biomarker is determined by immunohistochemistry staining for the biomarker in tissue sections.

120. The method of any one of claims 18-20, wherein the subject is prognosed as having a tumor when the H-score is greater than 100.

121. The method of claim 120, wherein the tumor comprises an adenocarcinoma.

122. The method of any one of claims 104-121, wherein the subject has been determined to have Lynch Syndrome.

123. The method of any one of claims 104-115, wherein the subject has not been diagnosed with Lynch Syndrome.

124. The method of any one of claims 104-123, wherein the subject has not been diagnosed with and/or has not been treated for colorectal cancer.

125. The method of any one of claims 104-124, wherein the method further comprises treating the subject.

126. The method of claim 125, wherein the subject is treated for stage I or stage II colorectal cancer.

127. The method of any one of claims 124-126, wherein the colorectal cancer comprises mismatch repair deficient colorectal cancer (MMR-d).

128. The method of any one of claims 104-127, wherein the expression level is normalized.

129. The method of any one of claims 104-128, wherein the sample from the subject is a sample from a primary colorectal cancer tumor.

130. The method of any one of claims 104-128, wherein the sample from the subject is a sample from a biopsy of colorectal tissues.

131. The method of any one of claims 104-130, wherein the sample from the subject is a sample of colon mucosa or a culture of cells derived from the colon mucosa of the subject.

132. The method of claim 131, wherein the sample comprises an organoid derived from the colon mucosa of the subject.

133. The method of any one of claims 104-132, wherein the expression levels of the one or more biomarkers in the subject is i) increased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

134. The method of any one of claims 104-133, wherein the expression levels of the one or more biomarkers in the subject is i) decreased compared to the levels of expression in samples from subjects identified as not having MMR-d colorectal cancer, identified as low risk, or in normal tissues or ii) within the range of expression levels in samples of subjects identified as having MMR-d colorectal cancer or identified as high risk.

135. The method of any one of claims 125-134, wherein the treatment comprises one or more of surgery, partial colectomy, surgical removal of lymph nodes, radiation, targeting therapy, adjuvant chemotherapy, and neo adjuvant chemotherapy.

136. The method of claim 135, wherein the treatment excludes one or more of surgery, partial colectomy, surgical removal of lymph nodes, radiation, targeting therapy, adjuvant chemotherapy, and neo-adjuvant chemotherapy.

137. The method of claim 136, wherein the treatment excludes chemotherapy, adjuvant chemotherapy, or neo-adjuvant chemotherapy.

138. The method of claim 137, wherein the chemotherapy comprises one or more of 5-FU, leucovorin, oxaliplatin, capecitabine, and irinotecan.

139. The method of any one of claims 135-138, wherein the targeted therapy comprises one or more of bevacizumab, ziv-aflibercept, ramucirumab, cetuximab, and panitumumab.

140. The method of any one of claims 135-138, wherein the targeted therapy excludes one or more of bevacizumab, ziv-aflibercept, ramucirumab, cetuximab, and panitumumab.

141. The method of any one of claims 125-140, wherein the treatment comprises one or more of regorafenib, trifluridine, and tipiracil.

142. The method of any one of claims 125-140, wherein the treatment excludes one or more of regorafenib, trifluridine, and tipiracil.

143. The method of any one of claims 104-142, wherein measuring the level of expression of one or more biomarkers comprises measuring the amount of protein produced from the biomarker gene.

144. The method of any one of claims 104-142, wherein measuring the level of expression of one or more biomarkers comprises measuring the amount of mRNA produced from the biomarker gene.

145. The method of any of claims 104-144, wherein the subject has undergone surgery to resect all or part of the cancer.

146. The method of any one of claims 104-145, wherein the subject has not undergone surgical resection of the tumor.

147. The method of any of claims 104-146, wherein the sample comprises a pre-operative and/or post-operative.

148. The method of any one of claims 133-147, wherein low risk is indicative of a subject with a low risk for distant metastasis and good overall survival (OS) rate, and high risk is indicative of a subject with a high risk for distant metastasis and poor overall survival (OS) rate.

149. The method of any one of claims 104-148, wherein the method further comprises evaluating the sample from the subject for MMR-d and/or microsatellite instability (MSI).

150. The method of any one of claims 104-149, wherein the sample is from a subject that has been evaluated for MMR-d and/or MSI.

151. The method of claim 149 or 150, wherein evaluating the sample from the subject for MMR-d comprises determining the level of expression of one or more of MLH1, MSH2, MSH6, PMS2, and EPCAM.

152. The method of any one of claims 149-151, wherein the sample is from a subject has been determined to be MMR-d and/or MSI+.

153. The method of any one of claims 104-152, wherein the cancer comprises stage 0, I, II, III, or IV cancer.

154. The method of any one of claims 104-153, wherein the cancer excludes stage 0, I, II, III, or IV cancer.

155. A kit comprising 1, 2, 3, 4, or 5 detection agents for determining expression levels of biomarkers for colorectal cancer, wherein the biomarkers comprise one or more biomarker genes from Table 1, Table 2, Table 3, Table 4, Table 5, Table S2, Table S3, Table S4, Table S5, FIG. 4A, and/or FIG. 4B.

156. The kit of claim 155, wherein the kit further comprises one or more negative or positive control samples and/or control detection agents.
